(19) United States
(12) Patent Application Publication
(10) Pub. No.: US 2011/0158969 A1
(43) Pub. Date: Jun. 30, 2011

(54) COMPOSITIONS AND METHODS FOR USING STROMAL CELLS TO ENHANCE TREATMENT OF CENTRAL NERVOUS SYSTEM INJURIES

(75) Inventor: Michael Chopp, Southfield, MI (US)

(73) Assignee: HENRY FORD HEALTH SYSTEM, Detroit, MI (US)

(21) Appl. No.: 12/920,277

(22) PCT Filed: Feb. 24, 2009
(86) PCT No.: PCT/US09/34997

§ 371 Date: Mar. 3, 2011

Related U.S. Application Data
(60) Provisional application No. 61/032,365, filed on Feb. 28, 2008.

(51) Int. Cl. A61K 35/28; A61K 35/12; A61P 25/28

(52) U.S. Cl. 424/93.21; 424/93.7

(57) ABSTRACT

The present invention provides novel methods and compositions for the treatment of injuries to the mammalian central nervous system. These methods involve administering stromal cells in combination with a blood-brain barrier permeabilizing agent in order to enhance neurorestoration, functional neurological recovery, stromal cell engraftment, and treatment of neurodegenerative diseases.

### STATEMENT OF GOVERNMENT INTEREST

This invention was made with government support under Grant No. NS042345 awarded by the NIH-NINDS. The government has certain rights in this invention.

### BACKGROUND OF THE INVENTION

1. Field of the Invention

The field of the present invention is generally related to the treatment of an injured central nervous system. More, specifically, the present invention is directed to the treatment of an injured central nervous system by administering stromal cells and a blood-brain barrier permeabilizing agent.

2. Description of the Related Art

Most central nervous system (CNS) injuries are caused by stroke, traumatic brain injury, spinal cord injury, hypoxia-ischemia, seizure, infection, and poisoning, all of which may directly or indirectly cause a disruption of blood supply to the CNS. These injuries commonly lead to ischemia, irreversible brain and/or spinal cord damage, apoptosis of the injured CNS tissue, and in some instances, death of the injured individual.

Stroke is the third leading cause of death in developed countries. Stroke is one of the leading contributors to adult disability and affects roughly 40 million people worldwide. The nature of alterations in cerebrovascular structures that contribute to stroke include blood clots that form in the blood vessels of the brain (thrombus), blood clots or pieces of atherosclerotic plaque or other material that travel to the brain from another location (emboli), and hemorrhage of blood vessels. Thus, stroke may be caused by reduced blood flow as a result of a cerebrovascular hemorrhage or clot, thereby resulting in deficient blood supply, ischemia, and/or infarction of the injured tissue.

Hemorrhagic stroke, which is also known as intracerebral hemorrhage (ICH), causes 10% to 15% of strokes, with a thirty day mortality rate of 35% to 52%; half of the deaths occur within the first two days (Broderick JP, Brott T, Tomsick T, Miller R, Huster G. J Neurosurgery. 1993; 78:188-191; Anderson C S, Chakera T M, Stewart-Wynne E G, Jamrozik K D. J Neurol Neurosurg Psychiatry. 1994; 57:936-940; Counsell C, Boonyakarnkul S, Dennis M, Sandercock P, Bamford J, Cerebrovasc Dis. 1995; 5:26-3.). Of the estimated 67,000 patients who had an ICH in the United States during 2002, only 20% were expected to be functionally independent at 6 months post-injury (Counsell C, Boonyakarnkul S, Dennis M, Sandercock P, Bamford J, Cerebrovasc Dis. 1995; 5:26-3.).

Substantial ongoing bleeding occurs in patients with ICH, particularly during the first 3 to 4 hours after onset, which is linked to neurological deterioration in these patients. Moreover, one study demonstrated that stroke patients often do not even receive medical attention until 3-6 hours after the stroke (Evenson et al., 2001 Neuroepidemiology, 20(2): 65-76). Normally, acute stroke treatments must be administered within the first few hours after the insult in order to be effective, and they do not provide any neurorestorative effects in the injured CNS tissue. Thus, if the time window for effective stroke treatment could be extended beyond that currently available for acute neuroprotective stroke treatments, (i.e., perhaps days or weeks, rather than minutes or hours after stroke) there would be an opportunity to treat most, if not all, stroke patients. Such treatments would also enhance the neurorestoration and functional neurological recovery in these patients. In view of these observations, there is currently an urgent need to search for novel cellular and pharmacological therapeutic approaches that can are effective beyond the hyper-acute phase of ischemia. Amplifying the intrinsic properties of the brain for neuroplasticity and subsequent neurological recovery becomes critical in post-stroke recovery treatments designed to enhance structural and functional reorganization (i.e., plasticity) of the damaged brain.

Intracerebral transplantation of donor stem cells from embryonic tissue have been shown to differentiate into neural cells (Snyder et al., 1997 Adv Neurol. 72:121-32). Intrastriatal fetal grafts have been used to reconstruct damaged basal ganglia circuits and to ameliorate behavioral deficits in a mammalian model of ischemia (Goto et al., 1997 Exp Neural. 147:503-9). Fetal hematopoietic stem cells (HSCs) transplanted into the adult organism or adult HSCs transplanted into an embryo resulted in a chimera that reflected the endogenous cells within the microenvironment into which the cells were seeded (Geiger et al., 1998, Immunol Today 19:236-41). Another group found that pluripotent stem cells were harbored in the adult CNS, and the adult brain could form new neurons (Gage, 1998 Curr. Opin. Neurobiol. 8:671-6; Kempermann and Gage, 1998 Nat. Med. 4:555-7).

Neural stem cells are important cell therapeutic candidates for the treatment of stroke and other CNS diseases because of their ability to differentiate in vitro and in vivo into neurons, astrocytes and oligodendrocytes. The powerful multipotent potential of stem cells, in general, may make it possible to effectively treat diseases or injuries with complicated disruptions in neural circuitry, such as stroke, where more than one cell population is affected. Indeed, in recent years, much attention has been focused on the ability of undifferentiated pluripotent stem cells to improve experimental neurological conditions, including ischemic stroke, brain trauma, and spinal cord injury (Chopp et al., 2000; Li et al., 2000; and Mahmood et al., 2003). Specifically, human embryonic neural stem cells have been used in a collagenase model of ICH to restore neurological function and demonstrate migration of the cells to the site of hemorrhage. However, no currently available medical therapy has shown a consistent or unambiguous benefit in terms of improving or enhancing neurorestoration, functional neurological recovery, and engraftment of therapeutic cells.

Another potential avenue for cell-based therapy is human umbilical cord blood cells (HUCB), which have a relatively high percentage of hematopoietic stem cells, and have been used to treat ischemic stroke in animal models. Several groups have demonstrated that HUCB cells survived and migrated into the CNS of normal and diseased animals and have been shown to promote functional recovery in animal models of ischemic stroke, spinal cord injury, and Intracerebral hemorrhage (Chen et al., 2001 Stroke, 32(11): 2682-8; Lu et al., 2002 Cell Transplant, 11(3): 275-81; Saporta et al., 2003 J. Hematotherapy & Stem Cell Research, 12: 271-278). Although, HUCB cells have been used to treat ischemic stroke, spinal cord injury, and intracerebral hemorrhage, doubt still remains as to the long term efficacy of HUCB treatments and their potential to engraft and promote neurorestoration in the central nervous system.

Bone marrow stromal cells (BMSCs), which are also known as mesenchymal stem cells (MSCs), have the potential to be used for cell therapy (Pereira et al., 1995; Pereira et al., 1998; Pittenger et al., 1999; and Prockop et al., 2003). BMSCs have a capacity for self-renewal and differentiation in a variety of non-hematological tissues. The potential use BMSCs for repairing and remodeling injured brain tissues has been reported using different animal models of injury (Chopp et al., 2000; Mahmood et al., 2003; Li et al., 2001; Kopen et al., 1999; Li et al., 2002; and Li et al., 2000). BMSC therapy induces neurorestorative changes in the brain, which are reflective of several mechanisms of action.

In previous models of neurological injury, BMSCs have been shown to pass through the blood-brain barrier to target sites of brain lesions (Li et al., 2001; Mahmood et al., 2003; and Zhang et al., 2002). In neonatal mouse, BMSCs migrate widely throughout the developing brain and have shown the capacity to differentiate into neurons and astrocytes (Kopen et al., 1999). BMSCs infused systemically into rats preferentially migrate to the ischemic cortex (Eglitis et al., 1999).

In recent studies, human BMSCs have shown significant benefit in animal models of ischemic stroke and closed head injury (Li et al., 2002 and Mahmood et al., 2003). In these models of neural damage, BMSCs appear to have the capacity to induce endogenous brain-derived cells, such as neural stem cells from the subventricular zone, to participate in the restorative process. However, the ability of BMSCs to localize to a region of brain injury and increase local concentrations of growth factors such as nerve growth factor, glial derived nerve growth factor, brain derived nerve growth factor and vascular endothelial growth factor may also be of paramount importance (Villars et al., 2000; Li et al., 2002; and Lu et al., 2004). These growth factors support and amplify angiogenesis, neurogenesis, neuronal migration, and synaptic plasticity (Carmeliet et al., 2002 and Jin et al., 2002). Thus, BMSCs appear to behave as small biochemical and molecular factories and catalysts, producing and inducing within parenchymal cells many cytokines and trophic factors that enhance angiogenesis and vascular stabilization in the ischemic boundary (Chen et al., 2003). However, the art has failed to provide methods to improve the efficacy of BMSC treatment modalities.

Several groups have investigated various BMSC concentrations and routes of administration to demonstrate the therapeutic efficacy of BMSC treatment of neural injury. Investigations using the rat MCA occlusion model demonstrated that one million hBMSCs administered intravenously failed to show significant benefit in animal recovery, whereas two million hBMSCs injected intraarterially improved neurological function (Chen et al., 2003 and Li et al., 2001). Previous work in an experimental traumatic brain injury model (TBI) using intraarterial cell therapy provided a direct route of administration, but resulted in increased cerebral ischemia due to small vessel cerebrovascular thrombosis by the therapeutic cells themselves (Lu et al., 2001).

The application of MSCs to treat experimental ICH has been less extensively studied than for the treatment of ischemic stroke and TBI. Four doses of two million mesenchymal stem cells delivered through the carotid artery to treat collagenase-induced ICH improved motor function in rats (Ueda et al., 1998). In another ICH injury model, one group demonstrated that BMSCs localize around the ICH (e.g., the site of injury) and that features of active neurorestoration and neuroregeneration are present after intravenous (IV) administration of 3-8 million BMSCs (Seyfried et al., 2006). This study indicated that the number of hBMSCs that were recruited to the injured site reached a plateau after the intravenous infusion of 3 to 8 million BMSCs (Seyfried et al., 2006). Thus, single treatments that would be effective with fewer BMSCs are desired in the art, as delivering multiple administrations and/or large numbers of BMSCs carries the serious risk of causing additional cerebrovascular occlusions in the already injured brain microvasculature.

Although the administration of BMSCs results in functional neurological improvements in a damaged CNS, these improvements are only partial, leaving significant room for increments in the efficacy of using BMSCs for the treatment of ICH and injuries of the mammalian CNS. Thus, there is a pressing need in the art for methods and compositions to improve the cell-based therapies used to treat CNS injuries, in order to enhance neurorestoration, functional neurological recovery, and engraftment of therapeutic cells.

### BRIEF SUMMARY OF THE INVENTION

The present invention provides for administering therapeutic compositions comprising a cell-based therapeutic and a blood-brain barrier permeabilizing agent to a mammal having a central nervous system injury.

In one embodiment, the present invention provides a method of enhancing neurorestoration in an injured central nervous system tissue of a mammal having a central nervous system (CNS) injury, comprising parenterally administering to the mammal an effective amount of stromal cells and a blood-brain barrier (BBB) permeabilizing agent to the mammal. In related embodiments, the stromal cells are selected from the group consisting of: bone marrow stromal cells, adipose tissue-derived stromal cells, liver stromal cells, and Wharton's jelly stromal cells. In certain embodiments, the stromal cells are bone marrow stromal cells.

In related embodiments, the BBB permeabilizing agent is selected from the group consisting of: alkylglyerols, RMP-7, and mannitol. In particular embodiments, the BBB permeabilizing agent is mannitol.

In further related embodiments, the stromal cells and the BBB permeabilizing agent are administered intravascularly. In other related embodiments, the stromal cells are administered intraarterially and the BBB permeabilizing agent is administered intravenously. In particular related embodiments, the BBB permeabilizing agent is administered prior to or about the same time as the administration of the stromal cells.

In other related embodiments, the stromal cells and the BBB permeabilizing agent are administered after a central nervous system injury. In particular embodiments, the stromal cells and the BBB permeabilizing agent are administered more than 1, 2, 4, 8, or 12 hours after a central nervous system injury. In further related embodiments, the stromal cells and the BBB permeabilizing agent are administered from about 12 hours to about 1 month after a central nervous system injury. In certain embodiments, the stromal cells and the BBB permeabilizing agent are administered from about 12 hours to about 1 week after the central nervous system injury. In related embodiments, the stromal cells and the BBB permeabilizing agent are administered about 12 hours to about 48 hours after the central nervous system injury.

In certain related embodiments, the mammal is a human.

In other related embodiments, the central nervous system injury is selected from the group consisting of: stroke, traumatic brain injury, spinal cord injury, hypoxia-ischemia, seizure, infection, and poisoning. In other related embodiments, the central nervous system injury is ischemic or hemorrhagic stroke. In further related embodiments the central nervous system injury results from a disease, disorder, or condition of the central nervous system selected from the group consisting of: Tay-Sachs disease, Sandhoffs disease, Hurler's syndrome, Krabbe's disease, Parkinson's disease, Alzheimer's disease, amyotropic lateral sclerosis (ALS), Huntington's disease, epilepsy, multiple sclerosis, spinal muscle atrophy (SMA), Friedreich's ataxia, Down's Syndrome, Wemicke-Korsakoff syndrome, and Creutzfeldt-Jakob disease.

In certain embodiments, following administration of the stromal cells and the BBB permeabilizing agent, the injured central nervous system tissue has increased expression of synaptophysin, neuronal class III β-tubulin (TUJ1), and doublecortin (DCX1), as compared to an identically injured central nervous system tissue in a mammal that has not been administered stromal cells and a BBB permeabilizing agent.

In another embodiment, methods of the present invention provide for enhancing the cognitive and/or motor functional neurological recovery of a mammal having a central nervous system injury, such methods comprising parenterally administering stromal cells and a blood-brain barrier (BBB) permeabilizing agent to the mammal. In related embodiments, following administration of the stromal cells and the BBB permeabilizing agent, the cognitive and/or motor functional neurological recovery of the mammal is greater compared to the cognitive and/or motor functional neurological recovery of an identically injured mammal that has not been administered the stromal cells and the BBB permeabilizing agent.

In further related embodiments, the stromal cells are selected from the group consisting of: bone marrow stromal cells, adipose tissue-derived stromal cells, liver stromal cells, and Wharton's jelly stromal cells.

In other related embodiments, the BBB permeabilizing agent is selected from the group consisting of: alkylglyerols, RMP-7, and mannitol.

In certain related embodiments, the stromal cells and the BBB permeabilizing agent are administered intravascularly. In related embodiments, the stromal cells are administered intraarterially and the BBB permeabilizing agent is administered intravenously.

In further related embodiments, the BBB permeabilizing agent is administered prior to or about the same time as the administration of the stromal cells. In other related embodiments, the stromal cells and the BBB permeabilizing agent are administered more than 12 hours after a central nervous system injury. In certain embodiments, the stromal cells and the BBB permeabilizing agent are administered from about 12 hours to about 1 month after a central nervous system injury. In particular embodiments, the stromal cells and the BBB permeabilizing agent are administered from about 12 hours to about 1 week after the central nervous system injury. In more particular embodiments, the stromal cells and the BBB permeabilizing agent are administered about 12 hours to about 48 hours after the central nervous system injury.

In related embodiments, the central nervous system injury is selected from the group consisting of: stroke, traumatic brain injury, and spinal cord injury. In further related embodiments, the central nervous system injury is ischemic or hemorrhagic stroke.

In another embodiment, methods of the present invention provide for enhancing the engraftment of stromal cells in an injured central nervous system tissue of a mammal having a central nervous system injury, comprising parenterally administering an effective amount of stromal cells and a BBB permeabilizing agent to the mammal. In related embodiments, the number of stromal cells engrafted in the injured central nervous system tissue, following administration of the stromal cells and BBB permeabilizing agent, is greater compared to the number of stromal cells engrafted in an identically injured central nervous system tissue of a mammal that has not been administered stromal cells and a BBB permeabilizing agent.

In certain related embodiments, the stromal cells are selected from the group consisting of: bone marrow stromal cells, adipose tissue-derived stromal cells, liver stromal cells, and Wharton's jelly stromal cells.

In further related embodiments, the BBB permeabilizing agent is selected from the group consisting of: alkylglyerols, RMP-7, and mannitol.

In other related embodiments, the stromal cells and the BBB permeabilizing agent are administered intravascularly. In related embodiments, the stromal cells are administered intraarterially and the BBB permeabilizing agent is administered intravenously.

In further related embodiments, the BBB permeabilizing agent is administered prior to or about the same time as the administration of the stromal cells. In particular embodiments, the stromal cells and the BBB permeabilizing agent are administered more than 12 hours after a central nervous system injury. In related embodiments, the stromal cells and the BBB permeabilizing agent are administered from about 12 hours to about 1 month after a central nervous system injury. In further related embodiments, the stromal cells and the BBB permeabilizing agent are administered from about 12 hours to about 1 week after the central nervous system injury. In yet further related embodiments, the stromal cells and the BBB permeabilizing agent are administered about 12 hours to about 48 hours after the central nervous system injury.

In related embodiments, the central nervous system injury is selected from the group consisting of: stroke, traumatic brain injury, spinal cord injury, hypoxia-ischemia, seizure, infection, and poisoning. In particular related embodiments, the central nervous system injury is ischemic or hemorrhagic stroke.

In another embodiment, methods of the present invention provide for treating an injured central nervous system tissue of a mammal having a central nervous system injury, comprising parenterally administering an effective amount of stromal cells and a blood-brain barrier permeabilizing agent.

In certain embodiments, the stromal cells have been genetically modified. In related embodiments, the stromal cells have been genetically modified to increase the expression of a growth factor selected from the group consisting of: nerve growth factor, glial derived neurotrophic factor, ciliary neurotrophic factor, brain derived growth factor, platelet derived growth factor, fibroblast growth factor, and vascular endothelial growth factor. In particular embodiments, the stromal cells are selected from the group consisting of: bone marrow stromal cells, adipose tissue-derived stromal cells, liver stromal cells, and Wharton's jelly stromal cells.

In further related embodiments, the BBB permeabilizing agent is selected from the group consisting of: alkylglyerols, RMP-7, and mannitol.

In other related embodiments, the stromal cells and the BBB permeabilizing agent are administered intravascularly.

In particular related embodiments, the blood-brain barrier permeabilizing agent is administered prior to or about the same time as the administration of the stromal cells. In related embodiments, the stromal cells and the BBB permeabilizing agent are administered more than 12 hours after a central nervous system injury. In further related embodiments, the stromal cells and the BBB permeabilizing agent are administered from about 12 hours to about 1 month after a central nervous system injury. In other related embodiments, the stromal cells and the BBB permeabilizing agent are administered from about 12 hours to about 1 week after the central nervous system injury. In yet other related embodiments, the stromal cells and the BBB permeabilizing agent are administered about 12 hours to about 48 hours after the central nervous system injury.

In certain related embodiments, the central nervous system injury is selected from the group consisting of: stroke, traumatic brain injury, spinal cord injury, hypoxia-ischemia, seizure, infection, and poisoning. In particular embodiments, the central nervous system injury is ischemic or hemorrhagic stroke. In other related embodiments, the central nervous system injury results from a disease, disorder, or condition of the central nervous system selected from the group consisting of: Tay-Sachs disease, Sandhoff's disease, Hurler's syndrome, Krabbe's disease, Parkinson's disease, Alzheimer's disease, amyotropic lateral sclerosis (ALS), Huntington's disease, epilepsy, multiple sclerosis, spinal muscle atrophy (SMA), Friedreich's ataxia, Down's Syndrome, Wemicke-Korsakoff syndrome, and Creutzfeldt-Jakob disease.

In another embodiment, methods of the present invention provide for a composition comprising an effective amount of stromal cells and a BBB permeabilizing agent. In related embodiments, the stromal cells have been genetically modified. In further related embodiments, the stromal cells have been genetically modified to increase the expression of a growth factor selected from the group selected from: nerve growth factor, glial derived neurotrophic factor, ciliary neurotrophic factor, brain derived growth factor, platelet derived growth factor, fibroblast growth factor, and vascular endothelial growth factor.

### BRIEF DESCRIPTION OF THE SEVERAL VIEWS OF THE DRAWINGS

FIG. 1 provides the results of functional neurological tests. Quantitative bar graph results of neurological severity score (NSS) (right panel) and corner turn test (CTT) (left panel) of four groups (control, human primary fibroblasts (FB); mannitol (MT); human bone marrow stromal cells (hBMSC); combination treatment, hBMSC+MT) are presented.

FIG. 2 provides bar graphs of quantitative striatal tissue loss percentages in the ICH region relative to the contralateral normal region of four groups (control, human primary fibroblasts (FB); mannitol (MT); human bone marrow stromal cells (hBMSC); combination treatment, hBMSC+MT) are shown. Statistical significance level is: *P<0.05.

FIG. 3 provides representative Immunostaining and quantitative immunoreactivities of mAb 1281, BrdU, synaptophysin, TUJ1 and DCX of sections of control and combination treatment rat striatum. Quantitative immunoreactivities for all treatment groups are presented as bar graphs on the right side of each panel. Colocalization of BrdU and TUJ1 in a subpopulation of cells near the injured region of the combination group is presented in the bottom panel. Arrows indicate cells positively stained for both BrdU and TUJ1.

### DETAILED DESCRIPTION OF THE INVENTION

### A. Methods of Treating and Preventing Neurological Injury

The present invention is based, in part, on administering therapeutic compositions to an injured mammalian central nervous system (CNS). As used herein, the term “central nervous system” or “CNS” should be construed to include the brain and spinal cord of a mammal. The term also includes the olfactory and optic cranial nerves. Tissues of the CNS include, but are not limited to tissues of the brain, spinal cord, optic nerve, individual regions of the aforementioned tissues, and the neuronal and non-neuronal cells comprising the said tissues and regions.

The methods and compositions of the present invention allow for cell-based therapeutic compositions comprising to be effectively administered to a patient having an injured CNS during an extended time window following injury. Thus, the methods of the present invention provide the opportunity to effectively treat a greater number of patients than was previously thought possible. Further, the present invention provides methods and compositions that reduce the risk of cerebrovascular occlusions associated with intraarterial delivery of cell-based therapeutics by administering an agent that permeabilizes the blood-brain barrier in combination with the cell-based therapeutic.

According to the present invention, enhancing the safety and efficacy of cell-based therapies in an injured central nervous system is largely determined by the ability of the cell-therapeutic to penetrate the blood-brain barrier and enter the injured CNS tissues. Previous studies indicate that the number of hBMSCs recruited to an injured central nervous system site reach a plateau after intravenous infusion of 3 to 8 million hBMSCs, suggesting the blood-brain barrier may be one of the rate-limiting steps in hBMSCs reaching the injured site (Seyfried et al, 2006). The blood-brain barrier is a complex vascular structure composed of a continuous layer of endothelial cells that maintain tight junctions between themselves. The properties of the blood-brain barrier suggest that a highly selective exchange system has evolved between the blood and brain to provide a homeostatic environment for the brain in the normal physiological state. This controlled environment may be altered by an increase in permeability under physiological conditions like hypertension or by physical damage of the endothelial membranes occurring with pathological conditions such as trauma, ischemia, tumors, and allergic or inflammatory diseases. In addition, an increase in permeability of the blood-brain barrier can be caused by a release of chemical mediators such as bradykinins, serotonin, histamines, arachidonic acid, leukotrienes, and free radicals.

The use of chemical mediators that increase the permeability of BBB can be advantageous when employed to increase drug delivery into the brain parenchyma. In experimental and clinical applications, the synthetic nonapeptide and bradykinin analog, Cereport, previously referred to as RMP-7, was found to selectively increase drug delivery into brain tumors and to increase the permeability of the blood-ocular barrier to ganciclovir in guinea pigs. When administered by either intravenous or intracarotid routes, Cereport selectively opens the blood-brain barrier via stimulation of the β₂ subclass of receptors on the brain endothelium. This stimulation leads to a rapid, transient increase in free intracellular Ca²⁺, which in turn causes an increase in endothelial pore size. This effect is temporary (˜20 min) due to tachyphylaxis or desensitization of β₂ receptor stimulation.

Another compound that may improve the permeability of the blood-brain barrier and that could offer a potential avenue of improvement in cell-based therapies of neurological injury is mannitol. For example, mannitol is a sugar alcohol and an osmolyte that has been used to prevent or treat medical conditions that are caused by an increase in body fluids/water (Winkler and Munoz-Ruiz, 1995). As an adjunctive treatment, mannitol has been frequently used to decrease edema or intracranial pressure with massive brain lesions (Schwarz et al., 1998 and McGraw and Howard, 1983). Mannitol has also be used to open the blood-brain barrier by temporarily shrinking the tightly coupled endothelial cells that make up the barrier, thus allowing for drugs to be delivered directly to the brain (Kroll and Neuwelt, 1998).

Another suggested mechanism of mannitol's effect on cerebral vasculature is increased endothelial permeability and small vessel dilation (Machi et al., 1996). Rheology of cerebral blood flow can be improved by mannitol, as it lowers the viscosity and allows better capillary flow (Burke et al., 1981). Mannitol can prevent the swelling of cells and alleviate the subsequent cell damage at the vicinity of the injured site (Tranum-Jensen et al., 1981). Although it is still somewhat controversial, high doses of mannitol have been reported to treat acute traumatic brain injury and reduce elevated intracranial pressure (Cruz et al., 2001 and Tranum-Jensen et al., 1981).

It is, therefore, a possibility that mannitol may enhance tissue tolerance to acute stress after injury and that mannitol may salvage more viable cells at the vicinity of the injury site (Lizasoain et al., 2006). In this way, mannitol may attenuate the stroke shock and therefore prolong the survival time window of injured tissues (Chen, et al. 2008). However, the use of agents such as mannitol has been poorly studied as a treatment for ICH, or as an adjunctive modality to cell-based therapeutics for injured CNS tissues.

Thus, in various embodiments, the present invention provides for parenterally administering stromal cells and a blood-brain barrier permeabilizing agent to a mammal having an injured CNS for enhancing neurorestoration and functional neurological recovery, enhancing engraftment of BMSCs in an injured CNS, decreasing tissue loss associated with CNS injury, and activating more endogenous cells in an injured CNS to increase synaptogenesis, immature neuron formation, and neuronal migration.

In particular embodiments, the stromal cells are selected from the group consisting of BMSCs, adipose-tissue derived stromal cells (ADSCs); liver stromal cells (LSCs); and Wharton's jelly stromal cells.

In various embodiments, the blood-brain barrier permeabilizing agent is selected from the group consisting of mannitol, RMP-7, and other suitable alkylglycerols.

In particular embodiments, the blood-brain barrier permeabilizing agent may be administered at about the same time or before the stromal cells in a separate or the same composition. In particular embodiments, compositions of the present invention, are administered to an individual after an injury to the CNS. In particular embodiments, compositions of the present invention, are administered to an individual with an injured CNS at least 1 hour, at least 2 hours, at least 3 hours, at least 4 hours, at least 5 hours, at least 6 hours, at least 7 hours, at least 8 hours, at least 9 hours, at least 10 hours, at least 12 hours, at least 24 hours, at least 48 hours, at least 72 hours, at least 1 week, at least 2 weeks, at least 3 weeks, and at least 1 month after the onset of the CNS injury. In certain embodiments, both the stromal cells and blood-brain barrier permeabilizing agent are administered between about 1 week and 1 month, between about 12 hours and 1 month, between about 12 hours and 2 weeks, between about 12 hours and 1 week, between about 12 hours and 72 hours, between about 12 hours and 48 hours, or between about 12 hours and 24 hours after the onset on the CNS injury.

The methods of the present invention contemplate parenteral intravenous and intraarterial as well as other appropriate parenteral routes), intrathecal, intraventricular, intraparenchymal (including into the spinal cord, brainstem or motor cortex), intracisternal, intracranial, intrastriatal, or intranigral administration of stromal cells in combination with a blood-brain barrier permeabilizing agent.

In particular embodiments, the administration of both the stromal cells and the blood-brain barrier permeabilizing agent may be by an intravenous or intraarterial route. In particular embodiments, stromal cells are administered via a different route than the blood-brain barrier permeabilizing agent. In certain embodiments, a blood-brain barrier permeabilizing agent is administered intravenously and before, concurrent with, or after the intraarterial administration of an effective amount of stromal cells to an individual with an injured CNS. In other embodiments, a blood-brain barrier permeabilizing agent is administered intraarterially and before, concurrent with, or after the intraarterial administration of stromal cells to an individual with an injured CNS. In other related embodiments, a blood-brain barrier permeabilizing agent is administered intravenously and before, concurrent with, or after the intravenous administration of stromal cells to an individual with an injured CNS. In yet other embodiments, a blood-brain barrier permeabilizing agent is administered intraarterially and before, concurrent with, or after the intravenous administration of stromal cells to an individual with an injured CNS.

In various embodiments, the stromal cells administered in a method including the administration of a blood-brain barrier permeabilizing agent is the same as or less than the amount of stromal cells administered to an identically injured CNS without the blood-brain barrier permeabilizing agent in order to achieve the same therapeutic benefits. In particular embodiments, the number of an effective amount of stromal cells administered in combination with a blood-brain barrier permeabilizing agent is less than 1×10¹² cells per 100 kg, less than 1×10¹¹ cells per 100 kg, less than 1×10¹⁰ cells per 100 kg, less than 1×10⁹ cells per 100 kg, less than 1×10⁸ cells per 100 kg, less than 1×10⁷ cells per 100 kg, less than 5×10⁶ cells per 100 kg, less than 4×10⁶ cells per 100 kg, less than 3×10⁶ cells per 100 kg, less than 2×10⁶ cells per 100 kg, less than 1×10⁶ cells per 100 kg, less than 5×10⁵ cells per 100 kg, less than 4×10⁵ cells per 100 kg, less than 3×10⁵ cells per 100 kg, less than 2×10⁵ cells per 100 kg, less than 1×10⁵ cells per 100 kg, less than 5×10⁴ cells per 100 kg, or less than 1×10⁴ cells per 100 kg.

In various embodiments the methods and compositions of the present invention enhance neurorestoration in an injured mammalian CNS or CNS tissue. As used herein, the terms “neurorestoration”, or “neurorestorative” describe events including synaptic plasticity (e.g., synaptogenesis), formation of immature neural cells (e.g., neurogenesis), angiogenesis, neuronal migration, as well as white matter and axonal remodeling, all of which can contribute to functional neurological improvement in an injured CNS. Without wishing to be bound to one particular theory, it is understood that injured central nervous system tissue, in many ways, recapitulates ontogeny (Cramer et al., 2000 and Goldman et al., 1996). For example, after stroke and other central nervous system injuries, cerebral tissue reverts to an earlier stage of development, and thus, becomes highly responsive to stimulation by cytokines, trophic factors, and growth factors.

In certain embodiments, enhanced neurorestoration is accomplished by methods of the present invention, in part, by inducing the expression of growth factors, such as brain derived nerve growth factor (NGF), glial derived neurotrophic factor (GDNF), ciliary neurotrophic factor (CTNF), brain derived growth factor (BDNF), platelet derived growth factor (PDGF), fibroblast growth factor (FGF), and vascular endothelial growth factor (VEGF), and other cytokines within the endogenous cells of the injured CNS by administering the therapeutic cell-based compositions of the present invention. Thus, in certain embodiments, “enhancing neurorestoration” is associated with increasing the rates or amount of cells undergoing synaptogenesis, neurogenesis, angiogenesis, and/or neuronal migration in an injured CNS due to the administration of a cell-based therapeutic, e.g., stromal cells and a blood-brain barrier permeabilizing agent, relative to either an untreated or control treated non-injured or injured CNS.

In particular embodiments, enhanced neurorestoration in an injured central nervous system tissue of a mammal is demonstrated by increased expression of synaptophysin, neuronal class III β-tubulin (TUJ1), and doublecortin (DCX1), compared to an identically injured central nervous system tissue in an individual that has not been administered the stromal cells and the blood-brain barrier permeabilizing agent. The skilled artisan would understand that indicators of neurorestoration include increased expression of genes such as synaptophysin, neuronal class III β-tubulin (TUJ1), and doublecortin (DCX), among others. Synaptophysin expression is indicative of synaptogenesis; TUJ1 is expressed in immature neurons and neuronal precursor cells; and DCX is expressed in migrating neurons.

The terms “immature neurons” and “neuronal precursor cells” are generally used interchangeably in many aspects of the present invention. Immature neurons may further be detected by the expression of one or more of the neural/neuronal phenotypic markers such as Musashi-1, Nestin, NeuN, class III β-tubulin, GFAP, NF-L, NF-M, microtubule associated protein (MAP2), S100, CNPase, glypican (especially glypican 4), neuronal pentraxin II, neuronal PAS 1, neuronal growth associated protein 43, neurite outgrowth extension protein, vimentin, Hu, internexin, O4, myelin basic protein and pleiotrophin, among others. Of course, one of ordinary skill in the art is able to recognize that there are many other “markers” which are commonly used to detect synapse formation, neurogenesis, and neuronal migration, each of which is suitable for use in determining neurorestoration.

Neurorestoration is an endogenous response that normally takes place in the CNS in response to injury. For example, after stroke in the adult brain, a neuroblast population marked by increased TUJ1 expression is greatly expanded in the subventricular zone (SVZ), and these cells are recruited to areas bordering the infarct, where they can differentiate into neurons and thereby replace lost neurons (Parent et al. Ann Neurol 2002; 52:802-813; Arvidsson et al. Nature Medicine 2002; 8: 963-970). An increase in neuronal cell migration is demonstrated by increased DCX expression. In addition, neuroblasts may act synergistically with the microvasculature to stimulate angiogenesis (marked by increased VEGF expression) and synapse formation (marked by increased synaptophysin and growth-associated protein 43 expression) in the local microenvironment and thereby promote neurorestoration and functional neurological recovery.

In other various embodiments, the methods of the present invention provide for enhancing the cognitive and/or motor functional neurological recovery of a mammal having an injured CNS. Injuries to the central nervous system are expected to negatively impact aspects of locomotor activity and/or cognitive ability depending on the nature of the injury. For example, in a middle cerebral artery occlusion model in rats, deficits in locomotor activity, neurological function, and cognitive performance were observed in experimental versus control groups (Borlognan et al., 1998; Roof et al., 2001).

Both cognitive and motor functional neurological recovery can be assayed using a variety of commonly practiced methods known to those of ordinary skill in the art in order to determine cognitive and motor functional neurological recovery. In rodents, for example, common cognitive tests of functional neurological recovery include the Morris Water Maze (MWM), passive avoidance tasks, the Y-maze/T-maze, fear conditioning, and object recognition tasks. Common tests used to measure functional neurological recovery in motor skills include the corner turn test (CTT), neurological severity score (NSS), open field locomotor activity test, rotarod test, grip strength assay, cat-walk gait analysis, balance beam test, and the inclined screen test. Similarly, one having ordinary skill in the art uses commonly practiced functional neurological assays in humans to determine the level of cognitive and motor functional neurological recovery in humans.

Other various embodiments of the present invention provide for methods of enhancing the engraftment of cell-based therapeutics e.g., stromal cells, in an injured mammalian CNS. As used herein, the term “engraft” or “engraftment” means the survival of a cell-based therapeutic in the injured CNS or CNS tissue, wherein the cell-based therapeutic remains present in the injured CNS for at least two weeks, at least one month, or at least one year after the administration of the therapy.

Without wishing to be bound to any particular theory, the present invention, contemplates, in part, that the enhanced engraftment of cell-based therapeutics leads to an increased efficacy of communication between therapeutic cells and endogenous cells. The increased level of cellular communication augments the endogenous cells' innate abilities to participate in the neuroregenerative process following an injury to the CNS. Thus, although it cannot be formally excluded that engrafted cells differentiate and replace the injured CNS cells, one of ordinary skill in the art recognizes that the increased neuroregenerative signals communicated by endogenous central nervous system cells is mediated by increased engraftment of cell-therapeutics and an important beneficial outcome of the therapeutic methods and compositions of the present invention.

### B. Diseases, Disorders, and Conditions of the Central Nervous System

The methods of the present invention may be used to enhance neurorestoration, functional neurological recovery, and cell-based therapeutic engraftment in mammals possessing one or more of a number of different types of injuries to the CNS including, sic, hemorrhagic stroke, ischemic stroke, traumatic brain injury, spinal cord injury, hypoxia-ischemia, infection, and poisoning. One of ordinary skill in the art would recognize that infant and adult onset genetic and/or neurodegenerative diseases also comprise injuries to the CNS, which can be effectively treated by the methods and compositions of the present invention.

Therapeutic compositions of the present invention can be administered to adults, and neonates and children having an injury of the CNS, including, for example, Tay-Sachs disease and the related Sandhoff's disease, Hurler's syndrome and related mucopolysaccharidoses and Krabbe's disease. With respect to adult diseases of the CNS, the methods and compositions of the present invention are useful for neurorestoration, functional recovery, and treatment of a variety of neurological diseases, including but not limited to, Parkinson's disease, Alzheimer's disease, amyotropic lateral sclerosis, Huntington's disease, epilepsy and the like. Treatment of multiple sclerosis is also contemplated.

Other neurodegenerative diseases and related injuries of the CNS that may be treated according to the present invention include, but are not limited to, AIDS dementia complex; demyelinating diseases, such as multiple sclerosis and acute transferase myelitis; experimental autoimmune encephalomyelitis (EAE); extrapyramidal and cerebellar disorders, such as lesions of the ecorticospinal system; disorders of the basal ganglia or cerebellar disorders; hyperkinetic movement disorders, such as Huntington's Chorea and senile chorea; drug-induced movement disorders, such as those induced by drugs that block CNS dopamine receptors; hypokinetic movement disorders, such as Parkinson's disease; progressive supra-nucleopalsy; structural lesions of the cerebellum; spinocerebellar degenerations, such as spinal ataxia, Friedreich's ataxia, cerebellar cortical degenerations, multiple systems degenerations (Mencel, Dejerine Thomas, Shi-Drager, and Machado-Joseph), systermioc disorders, such as Rufsum's disease, abetalipoprotemia, ataxia, telangiectasia; and mitochondrial multi-system disorder; demyelinating core disorders, such as multiple sclerosis, acute transverse myelitis; and disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Demetia of Lewy body type; Wemicke-Korsakoff syndrome; chronic alcoholism; Creutzfeldt-Jakob disease; Subacute sclerosing panencephalitis hallerrorden-Spatz disease; and Dementia pugilistica. see, e.g., Berkow et al., (eds.) (1987), The Merck Manual, (15.sup.th edition), Merck and Co., Rahway, N.J., which reference, and references cited therein, are entirely incorporated herein by reference.

### C. Cells of the Present Invention

The methods and compositions of the present invention provide for the administration of an effective amount of a cell-based therapeutic, e.g., stromal cells, in order to treat an injured CNS or CNS tissue. As used herein, the term “effective amount” includes those amounts of a cell-based therapeutic necessary to accomplish the intended function, e.g., enhancing neurorestoration, functional neurological recovery, or engraftment as described elsewhere herein. The effective amount will depend upon a number of factors, including the type of cell-based therapeutic used, age, body weight, sex, general health, severity of the condition to be treated, as well as the type and amount of blood-brain barrier permeabilizing agent that is administered with the cell-based therapeutic, e.g., stromal cells. The present invention contemplates that the effective amount of a cell-based therapeutic in a treatment including a blood-brain barrier permeabilizing agent as described herein will generally be less than the effective amount of cell-based therapeutic required to achieve the same degree of treatment in the absence of the blood-brain barrier permeabilizing agent.

In various embodiments, any type of cell may be used according to the present invention. In certain embodiments, cells of any mesodermal, endodermal, or ectodermal lineage may be administered in combination with a blood-brain barrier permeabilizing agent to a patient having an injured central nervous system.

In certain embodiments of the present invention, the preferred cell-based therapeutic is stromal cells. In particular embodiments of the present invention, the stromal cells are bone marrow stromal cells, adipose-tissue derived stromal cells (ADSCs); liver stromal cells (LSCs); or Wharton's jelly stromal cells. Stromal cells, also referred to as mesenchymal stem cells, are a mixed cell population that includes stem cells and progenitor cells. However, the term “stromal cells” should be reserved for a subset of these cells that demonstrate stem cell activity by clearly stated criteria (Horwitz et al., 2005. Clarification of the nomenclature for MSC: The International Society for Cellular Therapy Position Statement, Cytotherapy, 7, pp. 393-395).

In one embodiment, bone marrow stromal cells (BMSCs) are used as the cell-based therapeutic. As used herein, the terms “bone marrow stromal cells”, “BMSCs”, “marrow stromal cells”, “mesenchymal stem cells”, or “MSCs” are used interchangeably and refer to the small fraction of cells in bone marrow that can serve as stem cell-like precursors to osteocytes, chondrocytes, myocytes, adipocytes, and neuronal and non-neuronal cells of the central nervous system. BMSCs have been studied extensively (Castro-Malaspina et al., 1980, Blood 56:289-30125; Piersma et al., 1985, Exp. Hematol 13:237-243; Simmons et al., 1991, Blood 78:55-62; Beresford et al., 1992, J. Cell. Sci. 102:341-3 51; Liesveld et al., 1989, Blood 73:1794-1800; Liesveld et al., Exp. Hematol 19:63-70; Bennett et al., 1991, J. Cell. Sci. 99:131-139). BMSCs may be commercially obtained through various sources. For example, BMSCs isolated from human, mouse, rat, rabbit, dog, goat, sheep, pig, and horse are available from Cognate Bioservices Incorporated (Baltimore, Md.). Alternatively, BMSCs may be freshly isolated from any animal, by methods well known to those of ordinary skill in the art. In some embodiments, stromal cells are derived from mammals, and in particular embodiments, the stromal cells are derived from humans.

Sources of BMSCs and methods of obtaining and culturing BMSCs from those sources have been described in the art (e.g., Friedenstein et al., 1976 Exp. Hematol. 4: 267-274; Friedenstein et al., 1987, Cell Tissue Kinetics 20: 263-272; Castro-Malaspina et al., 1980, Blood 56: 289-301; Mets et al., 1981, Mech. Aging Develop. 16: 81-89; Piersma et al., 1985, Exp. Hematol. 13: 237-243; Owen et al., 1988, Cell and Molecular Biology of Vertebrate Hard Tissues, Ciba Foundation Symposium, Chichester, U. K., 42-60; Caplan, 1991, J. Orthoped. Res. 9: 641-650; Prockop, 1997, Science 276: 71-74; Beresford et al., 1992, J. Cell Sci. 102: 341-351; Cheng et al., 1994, Endocrinology 134: 277-286; Rickard et al., 1994, Develop. Biol. 161: 218-228; Clark et al., 1995, Ann. N.Y. Acad. Sci. 770:70-78). BMSCs can be obtained from substantially any bone marrow including, for example, bone marrow obtained by aspiration of the iliac crest of human donors. Methods for obtaining bone marrow from donors are well known in the art.

Stromal cells may be cultured in growth-promoting conditions, which can include any set of conditions (temperature, atmosphere, growth medium composition, humidity, degree of agitation, etc.) under which stromal cells normally proliferate. None of these conditions are critical. The temperature should be near that of normal human body temperature (i.e., about 37° C.), but can be any temperature at which stromal cells can proliferate (e.g., 30 to 43° C.). Stromal cells can be grown in an air atmosphere, or an air atmosphere supplemented with 5% CO2, for example. The growth medium can be any liquid medium which contains nutrients and factors sufficient to support proliferation of stromal cells. Such media contain, for example, a carbon source (e.g., glucose) and minimal essential nutrients, and preferably contain one or more of a mammalian serum (e.g., fetal calf serum), an antibiotic (e.g., penicillin or streptomycin), and L-glutamine (i.e., to improve amino acid supply for protein biosynthesis).

The mammalian serum can be used at a concentration of 1% to 20%, by volume, of the total growth medium. The serum is preferably pre-screened to ensure that it supports vigorous growth of stromal cells; some lots, even lots provided from the same supplier, do not support vigorous growth of stromal cells. Alternatively, the mammalian serum can be replaced with one or more growth factors (e.g., fibroblast growth factor, platelet derived growth factor, insulin growth factor, or endothelial growth factor). The growth medium can, for example, be Minimal Essential Medium-alpha without deoxyribonucleotides or ribonucleotides, supplemented with fetal calf serum, antibiotics, and L-glutamine; Dulbecco's minimal essential medium; and others well known to one of ordinary skill in the art. The growth medium is preferably replaced one or more times (e.g., every 3 or 4 days) during culture of the stromal cells.

One of ordinary skill in the art would appreciate, for example, that BMSCs can be expanded and simultaneously retain a pluripotent state (i.e., the ability to differentiate into one of numerous cells types, such as osteoblasts, adipocytes, and cells of the CNS, for example). Moreover, methods to differentiate BMSCs into various cell types in vitro have been described in the art (e.g., WO 96/30031, WO 99/43286, and U.S. Pat. No. 7,279,331).

Stromal cells administered in the methods of the present invention (e.g., BMSCs) can be cultured using art-known methods for a period of about 1 hour to 1 year. In some embodiments, stromal cells of the present invention can be maintained in culture for about 1 to 30 days, about 5 to 20 days, or about 3 to 14 days and are preferably harvested after not more than about 14 days, 10 days, or 7 days.

Stromal cells can be expanded by seeding the cells on a growth surface in the presence of a growth medium, and then harvesting the cells after, e.g., 10 days). Alternatively, the stromal cell expansion can be performed in series, meaning that the cells are expanded more than once. For example, after a first expansion on a first growth surface, stromal cells are harvested and then expanded in a growth medium on a second growth surface. Of course, the twice-expanded stromal cells can be harvested and subjected to one or more additional rounds of expansion, using the same method. There is no theoretical limit to the number of rounds of expansion and harvest that can be performed.

However, it is recognized that for most applications, no more than about 10 cycles of expansion and harvest for stromal cells will normally be necessary, and as few as 1, 2, 3, 4, or 5 cycles will be sufficient for many applications, including many of those described herein (e.g., use as a cell-based therapeutic for treatment of injured CNS or CNS tissue).

Additionally, one of ordinary skill in the art would recognize that methods for isolating the different types of stromal cells described herein are known in the art (e.g., ADSC's, Rodbell (1964) J Biol Chem 239:375 and Hanuer et al. (1989) J Clin Invest 84:1663-1670; LSCs, U.S. Patent App. Pub No. 2006/0057125; and Wharton's jelly stromal cells, McElreavey et al., 1991, Biochem. Soc. Trans. 636^{th} Meeting Dublin 19:29 S and U.S. Patent App. Pub No. 2004/0136967).

In some embodiments, the stromal cells that are to be introduced into a mammal may be derived from a different donor (allogeneic) or they may be stromal cells obtained from the individual to be treated (autologous). In addition, the stromal cells to be introduced into the individual can by obtained from an entirely different species (xenogeneic).

### D. Blood-Brain Barrier Permeabilizing Agents

One of ordinary skill in the art would understand that there are various blood-brain barrier permeabilizing agents known and commercially available in the art, all of which are suitable for use according to the methods of the present invention. For example, Cereport (e.g., RMP-7) is a commercial blood-brain barrier permeabilizing agent available from Alkermes Inc. (Cambridge, Mass.). In other particular embodiments, the blood-brain barrier permeabilizing agent is selected from the group consisting of RMP-7, mannitol, other suitable alkylglycerols, and phospho-derivatives of branched-chain lipophilic molecules (including those described in, for example, U.S. Pat. No. 7,186,703, which is incorporated herein by reference, in its entirety), among others. In particular embodiments, the blood-brain barrier permeabilizing agent is mannitol.

In related embodiments, the blood-brain barrier permeabilizing agent is administered at a concentration sufficient to increase the permeability of the blood-brain barrier. In particular embodiments, for example, wherein the blood-brain barrier permeabilizing agent is mannitol, the blood-brain barrier permeabilizing agent is administered at a concentration of about 0.25 g/kg to 3 g/kg, about 0.5 g/kg to 2.5 g/kg, about 1 g/kg to 2 g/kg, about 1.25 g/kg to 1.75 g/kg or about 1.5 g/kg. In particular embodiments, the blood-brain barrier permeabilizing agent (e.g., mannitol) is administered at a concentration greater than 0.10 g/kg, greater than 0.25 g/kg, greater than 0.50 g/kg, greater than 0.75 g/kg, greater than 1.0 g/kg, greater than 1.25 g/kg, greater than 1.50 g/kg, greater than 1.75 g/kg, or greater than 2.00 g/kg or more.

In other related embodiments, for example, wherein the blood-brain barrier permeabilizing agent is Cereport, the blood-brain barrier permeabilizing agent is administered at a concentration of about 0.01 μg/kg to 1 mg/kg, about 0.1 μg/kg to 100 μg/kg, or about 1 μg/kg to 10 μg/kg or any increment of concentration in between. For example, in particular embodiments, Cereport is administered at about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, or about 10 μg/kg.

In particular embodiments, Cereport is administered at a concentration greater than 0.005 μg/kg, greater than 0.01 μg/kg, greater than 1.0 μg/kg, greater than 10 μg/kg, greater than 50 μg/kg, greater than 100 μg/kg, greater than 250 μg/kg, greater than 500 μg/kg, or greater than 1000 μg/kg or more. As would be understood by one of ordinary skill art, the dosage of any particular blood-brain barrier permeabilizing agent may be determined using routine methods in the art. Additionally, the manufacturer's recommended dosage can be used to elicit the intended duration of blood-brain barrier permeability. The above described dosages are only examples and are not to be construed as limiting in this regard.

In particular embodiments, the blood-brain barrier permeabilizing agent induces a transient permeabilization of the blood-brain barrier. In related embodiments the duration of permeabilization is between about 1 minute and about 1 hour, about 2 minutes and 45 minutes, about 5 minutes and 30 minutes, about 10 minutes and 30 minutes, or about 15 minutes and 25 minutes.

In related embodiments, the transient permeability of the blood-brain barrier is maintained for about 1 minute, 2 minutes, 5 minutes, 10 minutes, 15 minutes, 20 minutes, 25 minutes, 30 minutes, 35 minutes, 40 minutes, 45 minutes, 50 minutes, 55 minutes, or 60 minutes or any duration of minutes in between.

### E. Enhancing Neurorestoration in an Injured CNS

Recent attention in neurorestorative treatments in an injured CNS has focused on using undifferentiated pluripotent stromal cells (e.g., BMSCs) in improving neurological outcomes following experimental neurological injury, including ischemic stroke, head injury, and spinal cord injury (Chopp et al., 2000; Eglitis et al., 1999; and Mahmood et al., 2003). However, the blood-brain barrier regulates entry of many blood-borne substances into the brain, and may exclude potentially therapeutic agents from entering the brain. Importantly, BMSCs have been shown to pass through the blood-brain barrier to target sites of brain lesions under experimental conditions (Prockop et al., 1997; Li et al., 2001; and Zhang et al., 2002). BMSCs have shown the capacity to differentiate into neurons and astrocytes and have the ability to preferentially migrate to damaged cortex (Kopen et al., 1999). Of particular significance is the BMSCs ability to secrete or stimulate secretion of growth factors which create a local environment conducive to neuroregeneration and neurorestoration (Chopp and Li, 2002).

In numerous reports, animals treated with human BMSCs have shown significant improvement after ischemic stroke and traumatic brain injury (Mahmood et al., 2003; Li et al., 2001; Li et al., 2002; Li et al., 2000; and Lu et al., 2004). Seyfried and colleagues have demonstrated the beneficial effects of hBMSC infusion in rats subjected to hemorrhagic stroke or intracerebral hemorrhage (ICH), as evidenced by reduced tissue loss, mitotic activity, immature neuron formation, synaptogenesis, and neuronal migration (Seyfried et al. 2006). However, obtaining the maximum therapeutic delivery of the hBMSCs using the lowest injection concentration is a clinically relevant concern. In order to maximize the therapeutic potential of a minimal amount of BMSCs, the present invention provides for the administration of a blood-brain permeabilizing agent in combination with the stromal cells.

Various embodiments of the present invention are directed to enhancing the neurorestoration in an injured mammalian CNS by amplification of the endogenous responses to central nervous system injury. In particular embodiments, this is accomplished by administering a combination of an effective amount of stromal cells and a blood-brain barrier permeabilizing agent, thus enhancing synaptogenesis, neurogenesis, and neuronal migration in an injured CNS. Thus, cell-based therapeutics that enhance one or more of these neurorestorative events can enhance neurorestoration and improve functional neurological recovery in an injured CNS. Furthermore, this neurorestorative response is potentiated by combining the administration of stromal cells with a blood-brain barrier permeabilizing agent.

In one embodiment, a method of enhancing neurorestoration in an injured CNS tissue of a mammal is achieved by the administration of an effective amount of stromal cells and a blood-brain barrier permeabilizing agent. As used herein, the terms “blood-brain barrier permeabilizer” and “blood-brain barrier permeabilizing agent” means a substance that is capable of disrupting the integrity of the blood-brain barrier. The methods of the present invention contemplate the disruption of the blood-brain barrier, in part, to facilitate an increased number of stromal cells and higher levels of neurotrophic growth factors to enter the brain, and thus, enhance the neurorestoration in an injured CNS of a mammal. In particular embodiments, the mammal is selected from the group consisting of a human, mouse, rat, rabbit, dog, goat, sheep, pig, and horse. In other embodiments, the mammal is a human.

### F. Administration of Cells of the Present Invention

In various embodiments of the present invention, a method of enhancing neurorestoration in an injured CNS of a mammal is achieved by the administration of an effective amount of stromal cells and a blood-brain barrier permeabilizing agent. In particular embodiments, the amount of stromal cells administered to a mammal having an injured CNS in order to achieve therapeutic efficacy is less than, about the same as, or greater than the amount of stromal cells that must be administered to a mammal having an identically injured CNS in order to achieve a therapeutic effect in a method that does not include the step of administering a blood-brain barrier permeabilizing agent. For example, a fewer number of cells may be administered, since the blood-brain barrier permeabilizing agent allows more cells to reach the injured CNS. In certain embodiments, a greater number of cells may be used in combination with a blood-brain barrier permeabilizing agent without negative side-effects (e.g., cerebrovascular occlusion).

In particular embodiments, the number of an effective amount of stromal cells administered in combination with a blood-brain barrier permeabilizing agent is at least or about 2-fold, 3-fold, 4-fold, 5-fold, 6-fold, 7-fold, 8-fold, 9-fold, or 10-fold less than the number of stromal cells administered in a method lacking the administration of a blood-brain barrier permeabilizing agent to a mammal having an identically injured CNS. In other embodiments, the number of an effective amount of stromal cells administered in combination with a blood-brain barrier permeabilizing agent is about the same to about 5-fold, about 2-fold to 4-fold, or about 2.5-fold to 3.5-fold less than the number of stromal cells administered in a method lacking the administration of a blood-brain barrier permeabilizing agent to a mammal having an identically injured CNS. In particular embodiments the number of an effective amount of stromal cells administered in combination with a blood-brain barrier permeabilizing agent is less than 99%, less than 95%, less than 90%, less than 80%, less than 70%, less than 60%, less than 50%, less than 40%, less than 30%, less than 20%, or less than 10% of the number of stromal cells administered in a method lacking the administration of a blood-brain barrier permeabilizing agent to a mammal having an identically injured CNS.

In some embodiments, the number of an effective amount of stromal cells administered to a mammal with an injured CNS is between about 1×10⁴ and about 1×10¹³ cells per 100 kg of mammal. In some embodiments, the number of an effective amount of stromal cells administered is between about 1×10⁶ and about 1×10⁹ cells per 100 kg or between about 1×10⁸ and about 1×10¹² cells per 100 kg. In some embodiments, the number of an effective amount of stromal cells administered is between about 1×10⁹ and about 5×10¹¹ cells per 100 kg. In some embodiments, the number of an effective amount of stromal cells administered is about 5×10¹⁰ cells per 100 kg. In some embodiments, the number of an effective amount of stromal cells administered is 1×10¹⁰ cells per 100 kg.

In particular embodiments, the number of an effective amount of stromal cells administered in combination with a blood-brain barrier permeabilizing agent is less than 1×10¹² cells per 100 kg, less than 1×10¹¹ cells per 100 kg, less than 1×10¹⁰ cells per 100 kg, less than 1×10⁹ cells per 100 kg, less than 1×10⁸ cells per 100 kg, less than 1×10⁷ cells per 100 kg, less than 5×10⁶ cells per 100 kg, less than 4×10⁶ cells per 100 kg, less than 3×10⁶ cells per 100 kg, less than 2×10⁶ cells per 100 kg, less than 1×10⁶ cells per 100 kg, less than 5×10⁵ cells per 100 kg, less than 4×10⁵ cells per 100 kg, less than 3×10⁶ cells per 100 kg, less than 2×10⁶ cells per 100 kg, less than 1×10⁵ cells per 100 kg, less than 5×10⁴ cells per 100 kg, less than 1×10⁴ cells per 100 kg, or less than 1×10³ cells per 100 kg. One of ordinary skill in the art would be able to use routine methods in order to determine the correct dosage of an effective amount of stromal cells for methods of the present invention.

In certain embodiments, it is advantageous to administer an effective amount comprising fewer stromal cells in combination with a blood-brain barrier permeabilizing agent as compared to a method that does not include co-administration of a blood-brain barrier permeabilizing agent, in order to reduce the risk of cell-based therapy cerebrovascular occlusions. One of ordinary skill in the art would understand that such occlusions would be detrimental to neurorestoration and functional neurological recovery in an injured CNS.

The methods of the present invention are useful for enhancing neurorestoration and functional neurological recovery well after the onset of the injury to the CNS. Typically, acute management of ischemic and hemorrhagic stroke occurs within a few hours of the insult and mainly results in neuroprotective effects. Furthermore, while it is contemplated that some acute management of neurological injuries may be necessary in certain instances, these treatments will not always serve to establish the long-lasting cellular changes in an injured CNS that are responsible for neurorestoration and functional neurological recovery, as provided by the methods of the present invention.

In one embodiment, a method of enhancing neurorestoration in an injured mammalian CNS is achieved by the administration of an effective amount of stromal cells and a blood-brain barrier permeabilizing agent, wherein both the stromal cells and the blood-brain barrier permeabilizing agent are administered more than after the onset of the CNS injury. In other related embodiments, both the stromal cells and blood-brain barrier permeabilizing agent are administered to an individual with an injured CNS at least 12 hours after a CNS injury. In other related embodiments, both the stromal cells and blood-brain barrier permeabilizing agent are administered to an individual with an injured CNS about at least 1 hour, at least 2 hours, at least 3 hours, at least 4 hours, at least 5 hours, at least 6 hours, at least 7 hours, at least 8 hours, at least 9 hours, at least 10 hours, at least 12 hours, at least 24 hours, at least 48 hours, at least 72 hours, at least 1 week, at least 2 weeks, at least 3 weeks, or at least 1 month after the onset of the CNS injury. Additionally, in other particular embodiments both the stromal cells and blood-brain barrier permeabilizing agent are administered between about 1 week and 1 month, between about 12 hours and 1 month, between about 12 hours and 2 weeks, between about 12 hours and 1 week, between about 12 hours and 72 hours, between about 12 hours and 48 hours, or between about 12 hours and 24 hours after the onset on the CNS injury. One of ordinary skill in the art would appreciate that the methods and compositions of the present invention may be practiced at any time after a CNS injury, including at least 12 hours following a CNS injury and still elicit the desired effects.

It is contemplated that because the timing of the administration of an effective amount of stromal cells and blood-brain barrier permeabilizing agent, in certain embodiments, is well after the window for acute treatment strategies of many CNS injuries, that many more patients will be candidates for treatment. Moreover, such late-stage treatment employed by particular methods of the present invention results in enhanced neurorestoration and functional neurological recovery, which is not seen in acute management strategies or other later stage therapies currently employed in the art.

The methods of the present invention contemplate, in part, that the administration of an effective amount of stromal cells and a blood-brain barrier permeabilizing agent need not take place at exactly the same time. In particular embodiments, the blood-brain barrier permeabilizing agent is administered before, concurrent with, or after the administration of stromal cells to an individual with an injured CNS. In particular embodiments, the blood-brain barrier permeabilizing agent is administered immediately before or about 30 minutes, 20 minutes, 10 minutes, 5 minutes, 2 minutes, 1 minute, or 30 seconds before the administration of stromal cells to an injured CNS. In certain embodiments, the blood-brain barrier permeabilizing agent is administered immediately before to about 30 minutes before the administration of stromal cells to an injured CNS, or any interval of time between about 0 and 30 minutes before stromal cell administration. In particular embodiments the blood-brain barrier permeabilizing agent is added about 24 hours, 18 hours, 12 hours, 6 hours, 3 hours, 2 hours, or 1 hour before the administration of stromal cells to an injured CNS.

The methods of the present invention contemplate the administration of an effective amount of stromal cells and a blood-brain barrier permeabilizing agent by various routes known to those of ordinary skill in the art. As used herein, the term “administration” or “administering” is used throughout the specification to describe the process by which stromal cells and a blood-brain barrier permeabilizing agent of the present invention are delivered to an individual with an injured CNS for therapeutic purposes.

Administration of the compositions of the present invention can be accomplished in a number of ways, including, but not limited to, parenteral (such term referring to intravenous and intraarterial as well as other appropriate parenteral routes), intrathecal, intraventricular, intraparenchymal (including into the spinal cord, brainstem or motor cortex), intracisternal, intracranial, intrastriatal, and intranigral, among others, which allow the stromal cells used in the methods of the present invention to ultimately migrate to the target site needed.

In particular embodiments, administration can be modified upon the disease or condition treated and may preferably be via a parenteral route, for example, intravenously, or intraarterially, or by direct administration into the affected tissue in the brain. For example in cerebrovascular injuries, the intraarterial route for delivery of stromal cells of the present invention is appealing from the theoretical perspective of maximizing the delivery of a given quantity of cells directly to the vascular territory of affected tissue. From the clinical standpoint, the intraarterial route is appealing since it is used extensively with other therapies, including chemotherapy, embolization of tumors and arteriovenous malformations, and endovascular treatment of intracranial arterial stenosis or acute thrombotic occlusion.

In particular embodiments, the methods of the present invention contemplate the administration of a relatively high dose of a blood-brain barrier permeabilizing agent, such as mannitol, supplied in the ranges described elsewhere, herein, which may beneficially influence stromal cell migration to the site of CNS injury, as well as reduce complications associated with intravascular administration of stromal cells, and thus, enhance neurorestoration and functional neurological recovery.

In one embodiment, a method of enhancing neurorestoration in an injured CNS tissue of a mammal is achieved by parenteral administration of an effective amount stromal cells and a blood-brain barrier permeabilizing agent. In related embodiments, the administration of both the stromal cells and the blood-brain barrier permeabilizing agent may be by an intravenous or intraarterial route. In particular embodiments, stromal cells are administered via a different route than the blood-brain barrier permeabilizing agent. One of ordinary skill in the art will understand that using different routes of administration for stromal cells and a blood-brain barrier permeabilizing agent does not alter the time of administration of a blood-brain barrier permeabilizing agent relative to the time of administration of the stromal cells.

For example, in certain methods of the present invention, a blood-brain barrier permeabilizing agent is administered intravenously and before, concurrent with, or after the intraarterial administration of an effective amount of stromal cells to an individual with an injured CNS. In other embodiments, a blood-brain barrier permeabilizing agent is administered intraarterially and before, concurrent with, or after the intraarterial administration of stromal cells to an individual with an injured CNS. In other related embodiments, a blood-brain barrier permeabilizing agent is administered intravenously and before, concurrent with, or after the intravenous administration of stromal cells to an individual with an injured CNS. In yet other embodiments, a blood-brain barrier permeabilizing agent is administered intraarterially and before, concurrent with, or after the intravenous administration of stromal cells to an individual with an injured CNS.

One having ordinary skill in the art would appreciate that the stromal cells, blood-brain barrier permeabilizing agents, effective amounts of stromal cells, timing and routes of stromal cell and blood-brain barrier permeabilizing agent administration, as well as dosages for cells and agents described herein for enhancing neurorestoration in an injured CNS of a mammal are appropriate for use with methods and compositions of the present invention that are generally directed to enhancing cognitive and motor functional neurological recovery, and enhancing the engraftment of stromal cells in an injured CNS of a mammal.

### G. Enhancing Functional Neurological Recovery in an Injured CNS

Other various embodiments of the present invention relate to methods of enhancing the cognitive and motor functional neurological recovery in a mammal having an injured CNS, by parental administration of an effective amount of stromal cells and a blood-brain barrier permeabilizing agent (e.g., mannitol). As used herein, the term “functional neurological recovery” or “cognitive and motor functional neurological recovery” means improving, as a result of the employing methods or compositions of the present invention, the cognitive skills or motor and/or locomotor activities in a mammal having an injured CNS. Improvement can be measured as the difference in any given functional neurological task before and after treatment. Thus, enhancing the cognitive and motor functional neurological recovery of a mammal having an injured CNS means an increase in the performance of a given functional neurological task, wherein an effective amount of stromal cells and a blood-brain barrier permeabilizing agent has been administered to the mammal relative to the performance of the task in a mammal having an identically injured CNS but which has not been administered stromal cells and a blood-brain barrier permeabilizing agent (i.e., stromal cells alone or a blood-brain barrier permeabilizing agent alone.

In related embodiments, the cognitive and motor functional neurological recovery is enhanced from about 1% to 100%, about 5% to 75%, about 10% to 60%, about 25% to 50%, or about 35% to 40%, wherein 100% represents normal levels of cognitive and motor neurological function present in a non-injured CNS of a mammal. In certain embodiments the cognitive and motor functional neurological recovery is enhanced about 1%, 2%, 5%, 10%, 15%, 20%, 25%, 30%, 35%, 40%, 45%, 50%, 55%, 60%, 65%, 70%, 75%, 80%, 85%, 90%, 95%, or 100% or any percentage recovery in between.

### H. Enhancing Stromal Cell Engraftment in an Injured CNS

Other various embodiments of the present invention relate to methods of enhancing the engraftment of stromal cells in an injured CNS tissue of a mammal by parental administration of the stromal cells and a blood-brain barrier permeabilizing agent, such as mannitol. As used herein, the term “engraftment” means the presence of administered stromal cells in an injured mammalian CNS about two weeks to about one year after the treatment was administered. Thus, enhancing the engraftment of stromal cells in an injured CNS means an increase in the number of administered stromal cells present in an injured mammalian CNS at a time between about two weeks to about one year following a treatment comprising stromal cells and a blood-brain barrier permeabilizing agent, relative to the number of administered stromal cells present in an injured CNS following treatment with stromal cells in the absence of a blood-brain barrier permeabilizing agent.

The present invention contemplates, in part, that enhancement of the number of engrafted stromal cells in an injured CNS tissue of a mammal will facilitate more neuroregeneration by the endogenous cells of the injured CNS tissue, and thus, benefit the mammal by enhancing the rate and/or magnitude of neurorestoration, functional neurological recovery, and neuroregeneration in said injured CNS.

Engrafted stromal cells may be detected using a number of techniques known to one of ordinary skill in the art. Proteins for tracking the integration, differentiation, and migration of genetically modified stromal cells in the injured central nervous system tissue of a mammal can include, but are not limited to green fluorescent protein (GFP), any of the other fluorescent proteins (e.g., enhanced green, cyan, yellow, blue and red fluorescent proteins; Clontech, Mountain View, Calif.), or other tag proteins (e.g., LacZ, FLAG, Myc, His6, V5 and the like).

Tracking the integration, differentiation, and migration of genetically modified stromal cells in the injured central nervous system tissue of a mammal is not limited to using detectable molecules expressed from a vector or virus. The migration, integration, and differentiation of stromal cells can be determined using a series of probes that would allow localization of transplanted bone marrow stromal cells. Such probes include those for human-specific Alu, which is an abundant transposable element present in about 1 in every 5000 base pairs, thus enabling the skilled artisan to track the progress of the transplanted cell. Tracking transplanted cells may further be accomplished by using antibodies or nucleic acid probes for cell-specific markers detailed elsewhere herein, such as, but not limited to, NeuN, MAP2, neurofilament proteins, and the like.

It would also be appreciated by one of ordinary skill in the art that any type of probe, antibody, marker, label, tag, nucleic acid, or protein or the like which can discriminate the stromal cells to be administered from the endogenous cells of the injured CNS of the mammal being administered the stromal cells may be used to quantify the engraftment of stromal cells of the present invention.

In particular embodiments, administration of an effective amount of stromal cells in combination with a blood-brain barrier permeabilizing agent enhances the engraftment of the stromal cells about 1.1-fold to 10-fold, about 1.2-fold to 5-fold, about 1.25-fold to 2.5-fold, or about 1.25-fold to 2-fold relative to The amount of engraftment observed when the stromal cells are administered without a blood-brain barrier permeabilizing agent. In certain embodiments, the engraftment of the stromal cells is enhanced at least 1.1-fold, 1.2-fold, 1.3-fold, 1.4-fold, 1.5-fold, 2-fold, 2.5-fold, 5-fold, 10-fold, or greater. In particular embodiments, the engraftment occurs in an injured CNS tissue or adjacent to an injured CNS tissue.

### I. Genetically Modified Stromal Cells Used in the Methods of the Present Invention

The methods and compositions of the present invention also provide for the administration of a blood-brain barrier permeabilizing agent in combination with stromal cells expressing an exogenous protein or molecule (e.g., for a therapeutic purpose or for a method of tracking their integration, differentiation, and migration in the injured central nervous system tissue of a mammal). Thus, the invention encompasses the use of stromal cells comprising an expression vector. Methods for the introduction of exogenous DNA into the stromal cells with concomitant expression of the exogenous DNA in the stromal cells such as those described for cells in general, for example, in Sambrook et al. (2001, Molecular Cloning: A Laboratory Manual, Cold Spring Harbor Laboratory, New York), and in Ausubel et al. (2007, Current Protocols in Molecular Biology, John Wiley & Sons, New York).

Means for introducing transgenes into cells are well known. A variety of methods for delivering and expressing a nucleic acid within a mammalian cell are known to those of ordinary skill in the art. Such methods include, for example, viral vectors, liposome-based gene delivery (WO 93/24640; Mannino Gould-Fogerite, BioTechniques, vol. 6(7):682-691 (1988); U.S. Pat. No. 5,279,833; WO 91/06309; Feigner, et al., Proc. Natl. Acad. Sci. USA, vol. 84:7413-7414 (1987); and Budker, et al., Nature Biotechnology, vol. 14(6):760-764 (1996)). Other methods known to the skilled artisan include electroporation (U.S. Pat. Nos. 5,545,130, 4,970,154, 5,098,843, and 5,128,257), direct gene transfer, cell fusion, precipitation methods, particle bombardment, and receptor-mediated uptake (U.S. Pat. Nos. 5,547,932, 5,525,503, 5,547,932, and 5,460,831). See also U.S. Pat. No. 5,399,346.

Widely used retroviral vectors include those based upon murine leukemia virus (MuLV), gibbon ape leukemia virus (GaLV), Simian Immunodeficiency virus (SIV), human immunodeficiency virus (HIV), and combinations thereof. See, e.g., Buchscher, et al., J. Virol., vol. 66(5):2731-2739 (1992); Johann, et al., J. Virol., vol. 66(5):1635-1640 (1992); Sommerfelt, et al., Virol., vol. 176:58-59 (1990); Wilson, et al., J. Virol., vol. 63:2374-2378 (1989); Miller, et al., J. Virol., vol. 65:2220-2224 (1991); PCT/US94/05700, and Rosenburg and Fauci, in Fundamental Immunology, Third Edition (Paul ed., 1993)).

AAV-based vectors are also used to transduce cells with target nucleic acids, e.g., in the in vitro production of nucleic acids and polypeptides, and in vivo and ex vivo gene therapy procedures. See West, et al., Virology, vol. 160:38-47 (1987); U.S. Pat. No. 4,797,368; WO 93/24641; Kotin, Human Gene Therapy, vol. 5:793-801 (1994); Muzyczka, J. Clin. Invest., vol. 94:1351 (1994), and Samulski (supra) for an overview of AAV vectors. Construction of recombinant AAV vectors is described in a number of publications, including Lebkowski, U.S. Pat. No. 5,173,414; Tratschin, et al., Mol. Cell. Biol., vol. 5(11):3251-3260 (1985); Tratschin, et al., Mol. Cell. Biol. Vol. 4:2072-2081 (1984); Hermonat and Muzyczka, Proc. Natl. Acad. Sci., USA, vol. 81:6466-6470 (1984); and Samulski, et al., J. Virol., vol. 63:03822-3828 (1989).

Gene therapy using genetically modified stromal cells offers several unique advantages over direct gene transfer into the body. First, the addition of the therapeutic transgene to the stromal cells takes place outside the patient, which allows the clinician an important measure of control because they can select and work only with those stromal cells that both contain the transgene and produce the therapeutic agent in sufficient quantity.

Thus, methods of the present invention also provide for the administration of stromal cells that when an isolated nucleic acid is introduced therein, and the protein encoded by the desired nucleic acid is expressed therefrom, where it was not previously present or expressed in the cell or where it is now expressed at a level or under circumstances different than that before the transgene was introduced, a benefit is obtained. Such a benefit may be therapeutic or may include the fact that there has now been provided a system wherein the expression of the desired nucleic acid can be studied in vitro in the laboratory or in a mammal in which the cell resides, a system wherein cells comprising the introduced nucleic acid can be used as research, diagnostic and therapeutic tools, and a system wherein mammal models are generated which are useful for the development of new diagnostic and therapeutic tools for selected disease states in a mammal.

Administration of a stromal cell expressing a desired isolated nucleic acid can be used to provide the product of the isolated nucleic acid to another cell, tissue, or whole mammal where a higher level of the gene product is useful to treat or alleviate a disease, disorder or condition associated with abnormal expression, and/or activity. Therefore, the invention contemplates the administration of a stromal cell expressing a desired isolated nucleic acid where increasing expression, protein level, and/or activity of the desired protein can be useful to treat or alleviate a disease, disorder or condition involving the CNS.

In addition, various embodiments of the present invention provide for methods of treating an injured CNS of a mammal by parental administration of an effective amount of stromal cells and a blood-brain barrier permeabilizing agent, such as mannitol. In certain embodiments of the present invention, it is contemplated that treating an injured CNS of a mammal with a blood-brain barrier permeabilizing agent such as mannitol and an effective amount of stromal cells genetically engineered to express various CNS growth factors, trophic factors and cytokines would further enhance the neurorestoration, neuroregeneration, and functional neurological recovery in the mammal when compared an identically injured CNS in a mammal in which the genetically modified stromal cells and a blood-brain barrier permeabilizing agent are not administered or wherein only stromal cells are administered.

It is further contemplated that the methods of the present invention utilizing genetically engineered stromal cells will be useful in treating virtually any disease, disorder, or condition of the central nervous system, as described elsewhere herein.

In certain embodiments, prior to the administration of an effective amount of stromal cells and a blood-brain barrier permeabilizing agent to a mammal having an injured central nervous system, the stromal cells may be genetically engineered to produce molecules such as trophic factors, growth factors, cytokines, neurotrophins, such as nerve growth factor, glial derived neurotrophic factor, ciliary neurotrophic factor, brain derived growth factor, platelet derived growth factor, fibroblast growth factor, and vascular endothelial growth factor, which are beneficial to cells which are already present in the CNS.

For example, stromal cells can be cultured and genetically engineered prior to their introduction into a mammal having an injured CNS. Engineered stromal cells are administered to a mammal having an injured CNS along with a blood-brain barrier permeabilizing agent such as mannitol to facilitate enhanced engraftment of the modified BMSCs. The increased enhanced engraftment of genetically engineered BMSCs would further enhance the neurorestoration, neuroregeneration, and functional neurological recovery in the mammal when compared an identically injured CNS in a mammal in which the genetically modified stromal cells and a blood-brain barrier permeabilizing agent are not administered.

### J. Cell-Based Compositions of the Present Invention

The present invention further provides for compositions that can be used to enhance the neurorestoration, functional neurological recovery, stromal cell engraftment, and provide treatment for an injured mammalian central nervous system as described throughout herein. Compositions of the present invention comprise effective amounts of genetically modified or unmodified stromal cells in addition to a blood-brain barrier permeabilizing agent. In particular embodiments, stromal cells are bone marrow stromal cells, adipose tissue-derived stromal cells, liver stromal cells, or Wharton's jelly stromal cells. In related embodiments, the blood-brain barrier permeabilizing agent is an alkylglycerol, RMP-7, or mannitol. One of ordinary skill in the art would immediately appreciate that concentration ranges for stromal cells and blood-brain barrier permeabilizing agents described elsewhere herein are suitable for compositions of the present invention.

In certain embodiments, compositions of the present invention comprise an effective amount of genetically modified or unmodified stromal cells and a blood-brain barrier permeabilizing agent, optionally in combination with a pharmaceutically acceptable carrier, additive or excipient. In certain aspects of the present invention, compositions comprising stromal cells and a blood-brain barrier permeabilizing agent can further comprise sterile saline, Ringer's solution, Hanks Balanced Salt Solution (HBSS), or Isolyte S, pH 7.4. Any of the compositions of the present invention can optionally comprise serum free cellular media.

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art.

### EXAMPLES

### Example 1

### The Preadministration of Mannitol Improved Intraarterial Delivery of hBMSCs and Significantly Improved Functional Neurological Recovery in a Rat Intracerebral Hemorrhage (ICH) Model

### Experimental Overview:

From previous studies, it was apparent that intravascular injection of 3 to 8 million hBMSCs significantly improved functional neurological recovery in a rat model of ICH (Seyfried et al., 2006). Subsequently, we discovered that co-administration of a blood-brain barrier permeabilizing agent, in this case, mannitol, and hBMSCs further improved the efficiency of intravascular MSC delivery (i.e., fewer injected cells were required to achieve the same therapeutic efficacy of 3 to 8 million hBMSCs administered in the absence of mannitol) after ICH and resulted in an improved functional neurological outcome when compared to control treatments.

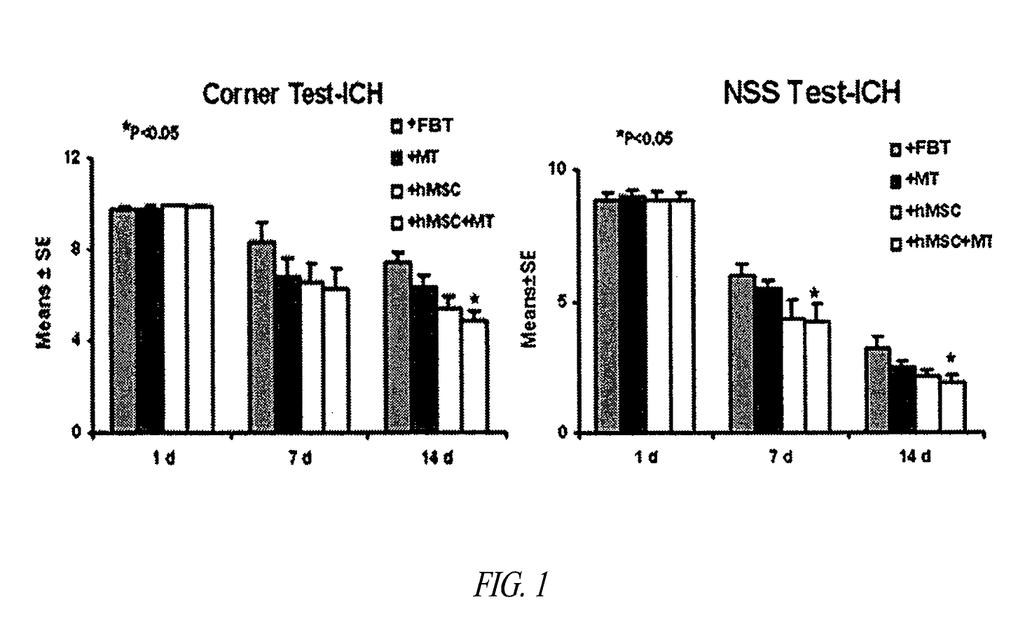

Co-administration of mannitol with hBMSCs significantly improved the functional neurological outcome in adult male Wistar rats subjected to experimentally induced ICH, in contrast to either treatment administered separately, or to the control administration of human fibroblasts. ICH was induced in 36 male Wistar rats by intrastriatal infusion of autologous blood. There were four post-ICH groups (N=9): group 1, negative control with only intraarterial injection of 1 million human fibroblasts; group 2, intravenous injection of mannitol; group 3, intraarterial injection of 1 million hBMSCs; group 4, intravenous injection of mannitol followed by intraarterial injection of 1 million hBMSCs. All animals survived the 2 week experimental period and functional outcome was measured using both neurological severity score (NSS) and corner turn testing score. FIG. 1 shows that only rats receiving the mannitol and hBMSCs combination treatment exhibited significant improvements in the corner turn and NSS tests.

The results of this experiment indicated that co-administration of mannitol increased the efficiency of low dose intraarterially administered hBMSCs. The therapy did not result in premature mortality and was an effective treatment for experimentally induced ICH. This study demonstrated that the combination therapy of mannitol and hBMSCs was more effective at increasing functional neurological recovery than either therapy alone when given intraarterially to treat ICH.

### Materials and Methods:

Animals and Reagents. Adult male Wistar rats were purchased from the Jackson Laboratory. All animal studies were carried out under the guidelines of the Institutional Animal Care and Use Committee (IACUC) of Henry Ford Health System. Human bone marrow stromal cells (hBMSCs) were provided by Cognate Therapeutics (Sunnyville, Calif.). Human primary fibroblasts were provided by Theradigm (Baltimore, Md.). Mannitol was obtained from Sigma (St. Louis, Mo.).

Animal surgical procedures of intravenous hemorrhage, intravenous infusion of mannitol and intraarterial infusion of hBMSCs. Thirty-six male Wistar rats weighing from 270 to 320 grams were used for intracerebral hemorrhage studies. Stereotactic stabilization and localization were used in the rats under general anesthesia as previously described (Seyfried et al., 2006). ICH was induced by injecting 100 μl of autologous blood, obtained from the femoral vein, into the right striatum at a steady infusion rate of 10 μl per minute (Seyfried et al., 2004). Twenty four hours after ICH, the animals were divided into four experimental groups. Group 1 received only intraarterial (via internal carotid artery) injection of 1 million human primary fibroblasts in phosphate-buffered saline (PBS) as a control. Group 2 received intravenous injection of mannitol at a dose of 1.5 g/kg in PBS via the tail vein. Group 3 received intraarterial (internal carotid artery) injection of 1 million hBMSCs in PBS. Group 4 received intravenous injection of mannitol at a dose of 1.5 g/kg followed 10 minutes later by intraarterial injection of 1 million hBMSCs in PBS. All treatments were administered 24 hours after induction of ICH. All rats also received a daily intraperitoneal injection of 100 mg/kg BrdU starting at day 1 after ICH for 14 days.

Functional neurological tests. Functional neurological outcomes were measured by the neurological severity score test (NSS) (Chen et al., 2001) and the corner turn test (Zhang et al., 2002), as previously described in Seyfried et al., 2006.

Statistical analysis. Statistical analysis of functional scores was performed using the Student's two-tailed t-test for independent samples. Data were presented as the mean±standard error and P values <0.05 were considered significant.

### Results:

Intravenous administration of mannitol followed by intraarterial injection of hBMSCs in an ICH animal model results in significantly improved functional neurological outcome. Four groups of adult male Wistar rats were treated as described in the Materials and Methods section and functional neurological outcome was assessed by NSS and corner turn test scores at 1, 7, and 14 days after ICH. All animals survived the 2-week experimental period. As shown in FIG. 1, at day 1 after ICH, just after the administration of the four classes of treatments, no apparent difference was observed among all the four groups by both NSS and the corner turn tests. Seven days after the experimentally induced ICH, functional neurological outcome measured by NSS started to show statistically significant improvement for the mannitol and hBMSC combination therapy group only (FIG. 1, right panel, group 4). At the end of second week after the ictus, the mannitol and hBMSC combination therapy group exhibited significantly improved functional neurological outcome assessed by NSS and corner tests (P<0.05) compared to the human fibroblast therapy control group (FIG. 1, compare groups 1 and 4). The individual mannitol and hBMSC therapy groups demonstrated a trend of improvement but failed to show any statistically significant neurological improvement (FIG. 1, groups 2 and 3).

Combination therapy of intravenous mannitol followed by low dose intraarterial hBMSC proved to be a safe and effective treatment in this rat ICH model as early as seven days after the ictus. The therapeutic treatment did not result in premature mortality and there was a significant functional benefit only when hBMSCs were preceded by mannitol. By administering mannitol prior to BMSCs, the significant improvement in functional neurological outcome, as measured by the NSS and corner turn tests, was accomplished with a much lower dose of BMSCs than previously described (Seyfried et al., 2006).

While therapies consisting of intraarterially administered hBMSCs have been effective in models of cerebral ischemia, it is likely that the benefit of mannitol in the ICH model is more apparent because of the differing nature of the inherent blood clot pathophysiology. A parenchymal hematoma creates an acute mass effect, compresses surrounding small vessels, and secretes vasogenic blood breakdown products; mannitol may counteract these effects while simultaneously improving the delivery of the cells (Boulard et al., 2003). These results have important ramifications for therapies targeting compromised vascular structures, or in situations where edema may limit the effectiveness of given therapy, wherein it may be advantageous to administer a smaller dose of BMSCs with mannitol to gain the same benefits that would be associated with higher doses of BMSCs, but without the caveats.

### Example 2

### Treatment with Mannitol and hBMSCs Significantly Reduced Tissue Loss in a Rat ICH Model Compared to Treatment with hBMSCs Alone

### Experimental Overview:

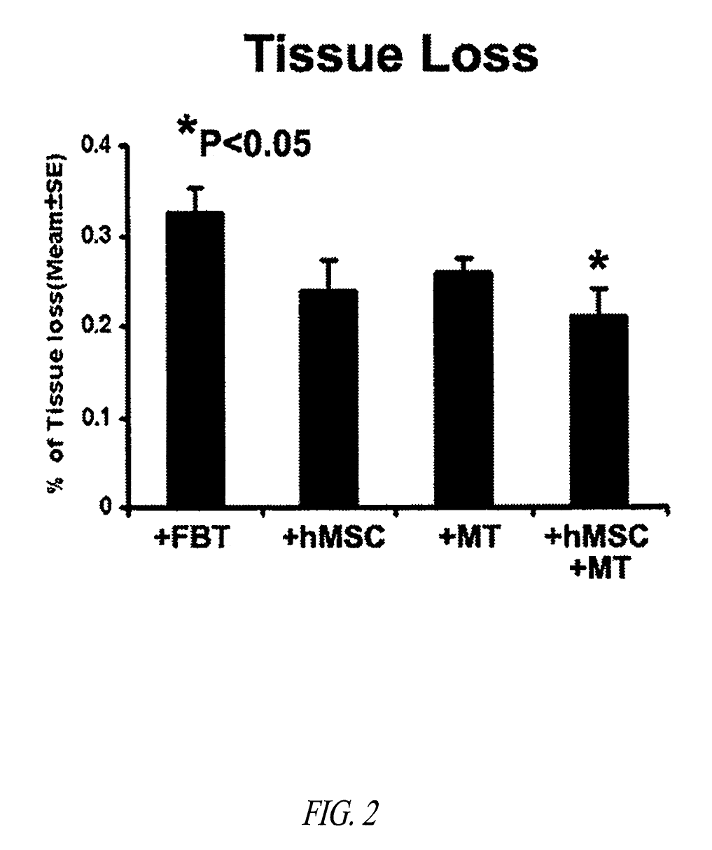

This experiment tested the hypothesis that co-administration of mannitol and hBMSCs would result in a statistically significant reduction in the degree of brain tissue loss in a rat ICH model. At fourteen days after the ictus, paraffin brain sections were prepared from the adult male Wistar rats used in Example I. Six sections from each rat brain were stained with hematoxylin and eosin (H & E) and the total number of cells was counted. The percentage of ipsilateral striatal tissue loss as a percentage of the untreated contralateral hemisphere was significantly reduced in the mannitol and hBMSCs combination treatment group compared to the other treatment groups (FIG. 2).

The results from these experiments demonstrated that the combination therapy of mannitol and hBMSCs not only provided improved functional neurological outcome, but significantly reduced tissue loss in a rat ICH model. Additionally, a significant reduction in brain tissue loss following ICH was only demonstrated for the combination therapy group (FIG. 2, group 4).

### Materials and Methods:

Histology and immunohistochemistry. At 14 days post-operation, the animals were anesthetized and perfused transcardially with 4% paraformadehyde in phosphate-buffered saline. Brain tissues were excised, fixed in formalin and sliced into 2 mm thick sections. Sections were embedded in paraffin and every 40^{th} coronal section, cut at a thickness of 6 μm between the bregma +0.1 mm to −0.86 mm of each rat brain for a total of six sections, was used for H & E staining and immunochemical staining. Percentage of the striatal tissue loss compared to the contralateral striatum was calculated using an image analysis system (Data Translation, Marlboro, Mass.).

Statistical Analysis. Statistical analysis of the area of ICH-related tissue damage was performed using the independent Student t-test. Data were presented as the mean±standard error and P values <0.05 were considered significant.

### Results:

The thirty six adult male Wistar rats that were the subject of the experiments in Example I, were further analyzed to examine the degree of brain tissue loss among treatment groups in the ICH affected regions. The percentage of tissue loss was notably reduced in the rats receiving the mannitol and hBMSC combination therapy with reference to the percentage of tissue loss in the other three groups.

The percentage of ipsilateral striatal tissue loss was calculated with reference to striatal tissue loss in the normal hemisphere. The actual percentage loss of striatal tissues on the side of hemorrhage is presented in FIG. 2 as follows: human fibroblasts=32.4±2.8%; 1 million hBMSCs alone=24±3.4% (P>0.05); mannitol alone=25.9±1.75% (P>0.05); and mannitol with hBMSCs=21±3.2% (P<0.01). Therefore, the percentage of striatal tissue loss was significantly reduced in the combination group when compared to the control groups. A trend of improvement was also observed when either the mannitol or hBMSCs was administered alone, but this trend was not statistically significant.

Combination therapy of mannitol followed by intraarterial hBMSC in this experiment demonstrated a significantly reduced amount of encephalomalacia, or tissue loss. This reduced tissue loss was only significant when the animals received mannitol and hBMSCs, and not when the animals received the intraarterial hBMSCs alone. These results highlighted the additional positive effects of a mannitol and hBMSCs combination therapy in reducing tissue loss in a neurological ischemia model.

### Example 3

### Immunostaining Perilesional ICH Rat Brain Sections Indicated Enhanced Neurorestoration in a Mannitol and hBMSCs Combination Treatment Group

### Experimental Overview:

This experiment tested the hypothesis that co-administration of mannitol and hBMSCs would in a statistically significant reduction in the degree of brain tissue loss in a rat ICH model. At fourteen days after the ictus, paraffin brain sections were prepared from the adult male Wistar rats in Example I. Six sections from each rat brain were hybridized to antibodies indicative of neurorestoration, as described below.

The results from these experiments demonstrated that the combination therapy of mannitol and hBMSCs enhanced the engraftment of hBMSCs in the ipsilateral rat striatum as evidenced by increased mAb 1281 staining, but also demonstrated increased neurorestoration as evidenced by increased staining for BrdU, synaptophysin, doublecortin, and neuronal β-tubulin isotype Ill.

### Materials and Methods

Reagents. 5′-bromo-2′ deoxyuridine (BrdU) was obtained from Sigma (St. Louis, Mo.). The following primary antibodies were used: monoclonal antibody against BrdU (1:100 Dako, Carpenteria, Calif.); synaptophysin (1:1,000 mAb, Clone SY 38; Chemicon. Temecula, Calif.); doublecortin (DCX) (1:50; Santa Cruz Biotechnology, Santa Cruz, Calif.), neuronal β-tubulin isotype III (TUJ1) (1:5,000 mAb; Covance, Berkeley, Calif.) and anti-Nuclei (1:500; specific for all human cell types; Chemicon. Temecula, Calif.)

Histology and immunohistochemistry. At 14 days post-operation, the animals were anesthetized and perfused transcardially with 4% paraformadehyde in phosphate-buffered saline. Brain tissues were excised, fixed in formalin and sliced into 2 mm thick sections. Sections were embedded in paraffin and every 40^{th} coronal section, cut at a thickness of 6 μm between the bregma +0.1 mm to −0.86 mm of each rat for a total of six sections, was used for H & E staining and immunochemical staining. Sections were blocked in a Tris-buffered saline containing 5% normal goat serum, 1% BSA and 0.05% Tween-20. Sections were then incubated with the primary antibodies for localization of BrdU (a marker for proliferation cells), TUJ1 (a marker for immature neurons), DCX (a marker for migrating neuroblasts, Feng et al., 2001) and the mAb 1281 (a marker specific for human nuclei, Mahmood et al., 2003). All immunostainings were performed at the same time with two negative controls of using the omission of primary antibody and the use of pre-immune serum for the quality control of the immunostaining procedure. For semiquantitative measurements of synaptophysin, TUJ1 and DCX, a series of six slides at various levels from the same block were used. Synaptophysin was measured in the striatum region. TUJ1 and DCX were measured at the subventricular zone. Synaptophysin, TUJ1 and DCX were digitized under a 20× objective lens (Olympus BX40; Olympus Optical Co, Tokyo, Japan) by using a 3-CCD color video camera (model DXC-970MD; Sony Corp, Tokyo, Japan) interfaced with an MCID image analysis system (Imaging Research, Inc, St. Catharines, ON, Canada). For synaptophysin, TUJ1 and DCX, data were presented as a percentage of the immunopositive areas in each field divided by the total areas in the field (628×480 μm²) (Chen et al., 2003). The number of BrdU-positive cells was counted in the boundary around the lesion. MAB1281 quantitative data were presented as the total number of MAB1281 immunoreactive cells within in the boundary around the lesion of each slide. Immunohistochemical analysis and the percentage of ipsilateral tissue loss were also performed to delineate proliferating immature neurons and neuronal migration.

Statistical Analysis. Statistical analysis of functional scores, area of ICH-related tissue damage, and histochemical results were all performed using the independent Student t-test. Data were presented as the mean±standard error and P values <0.05 were considered significant.

### Results:

Brain sections from thirty six adult male Wistar rats that were the subject of the experiments in Examples 1 and 2, were immunohistochemically stained for BrdU, mAb 1281, and expression of the genes: synaptophysin, TUJ1, and DCX.

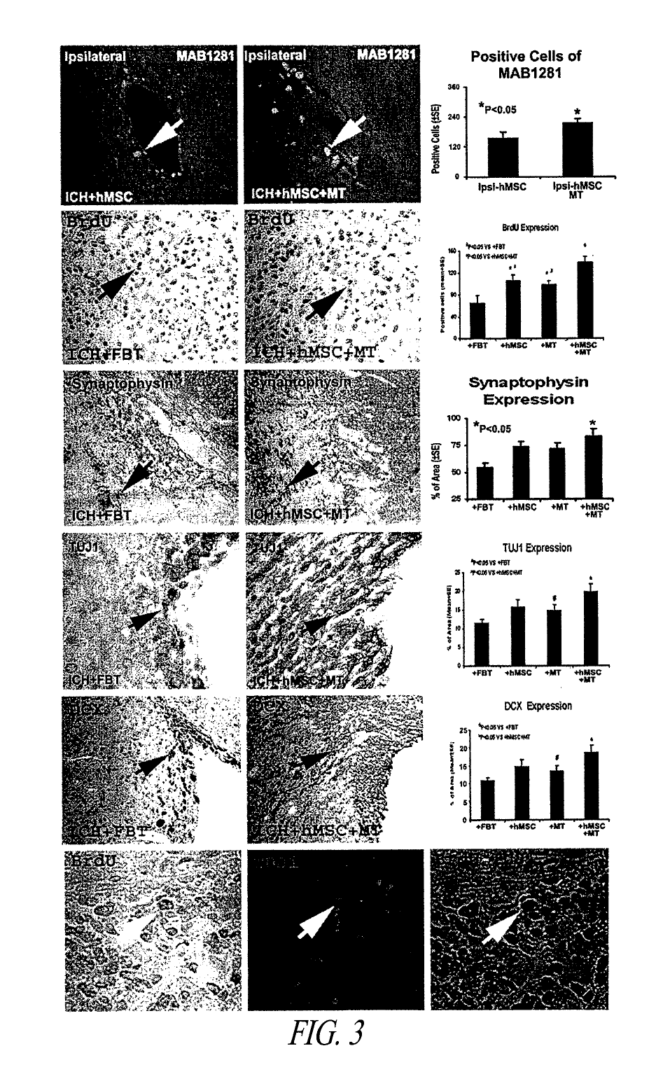

As shown in FIG. 3A, immunohistochemistry using mAb 1281 demonstrated that there were substantially more positive-staining cells (216±16, P<0.05) in the injured region of the combination group than that of the hBMSC group (157±21), suggesting that mannitol effectively potentiates hBMSCs to migrate to the injured site. Based on mAb 1281 immunostaining, the number of hBMSCs recruited to the injured region increased strikingly in the combination treatment group when compared to mannitol treatment alone.

Immunostaining against BrdU illustrated that there are significantly more BrdU positive staining cells in the boundary zone (P<0.05) around the injured site of the mannitol and hBMSCs combination treatment group compared to the control group (FIG. 3B). This increase in the number of BrdU positive cells suggests that mannitol and hBMSCs act synergistically to promote cell proliferation and migration to the injured region. One intriguing observation is that mannitol alone significantly increased the BrdU labeling in the vicinity of injury site (P<0.05), suggesting that mannitol itself may contain mitogenic activity to initiate DNA replication.

Immunohistochemical staining of the mannitol and hBMSC combination therapy group revealed significant increases in synaptophysin, TUJ1, and DCX expression (P<0.05) in the ICH affected regions compared to that of control group (FIG. 3, panels C, D, and E). Increased synaptophysin expression suggests increased synaptogenesis. An increase in TUJ1 expression is consistent with an increase in proliferating immature neurons, while an increase in DCX expression signifies an increase in neuronal migration. In order to detect if any new immature neurons are formed, co-immunostaining with both BrdU and TUJ1 was performed. As demonstrated in FIG. 3F, double staining for BrdU and TUJ1 revealed a sub-population of cells that expressed a neuronal marker while still dividing, suggesting that immature neurons were newly formed during the recovery stage in the combination treatment group.

Combination therapy of mannitol followed by intraarterial hBMSC in this experiment proved that the number of hBMSCs reaching the area of injury was significantly increased by mannitol infusion, and suggested that there was improved delivery through the microvasculature and/or better blood-brain barrier penetration. Additionally, mannitol was shown to be effective aid in the process of neurorestoration after ICH because there is an increase in the presence of BrdU positive cells in the affected ICH region compared to controls. Even though no statistically significant improvement of functional neurological outcome was noted when treating either with mannitol or hBMSCs alone, either mannitol alone or a low dose of hBMSCs alone significantly elicited cell proliferation at the injured site. This suggests that the combination treatment using hBMSCs and mannitol is truly synergistic and resulted in significant functional neurological recovery in this ICH model.

The beneficial effects of intraarterial infusion of MSCs were amplified with intravenous injection of mannitol. This lead to an increase in the number of cells with 5′-bromo-2′ deoxyuridine incorporation and the number of immature cells stained with antibodies to neuronal markers. Preadministration of mannitol significantly increased the number of hBMSCs located in the region of the ICH, improved histochemical parameters of neural regeneration and neurorestoration, and reduced anatomical and neuropathological consequences of ICH. This study further suggested that the combination therapy of mannitol and hBMSCs was more effective than either therapy alone when given intraarterially to treat ICH.

Furthermore, this study showed that treating injuries to the central nervous system far beyond the normal period of conventional acute management therapies was possible with the compositions of the present invention described herein, and were very effective at remodeling damaged central nervous system tissues.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of enhancing neurorestoration in an injured central nervous system tissue of a mammal having a central nervous system (CNS) injury, comprising parenterally administering to the mammal an effective amount of stromal cells and a blood-brain barrier (BBB) permeabilizing agent to the mammal.

2. The method of claim 1, wherein the stromal cells are selected from the group consisting of: bone marrow stromal cells, adipose tissue-derived stromal cells, liver stromal cells, and Wharton's jelly stromal cells.

3. (canceled)

4. The method of claim 1, wherein the BBB permeabilizing agent is selected from the group consisting of: alkylglyerols, RMP-7, and mannitol.

5. (canceled)

6. The method of claim 1, wherein the stromal cells and the BBB permeabilizing agent are administered intravascularly.

7. (canceled)

8. The method of claim 1, wherein the BBB permeabilizing agent is administered prior to or about the same time as the administration of the stromal cells.

9. The method of claim 1, wherein the stromal cells and the BBB permeabilizing agent are administered after a central nervous system injury.

10-13. (canceled)

14. The method of claim 1, wherein the mammal is a human.

15. The method of claim 1, wherein the central nervous system injury is selected from the group consisting of: stroke, traumatic brain injury, spinal cord injury, hypoxia-ischemia, seizure, infection, and poisoning.

16. (canceled)

17. The method of claim 1, wherein the central nervous system injury results from a disease, disorder, or condition of the central nervous system selected from the group consisting of: Tay-Sachs disease, Sandhoff's disease, Hurler's syndrome, Krabbe's disease, Parkinson's disease, Alzheimer's disease, amyotropic lateral sclerosis (ALS), Huntington's disease, epilepsy, multiple sclerosis, spinal muscle atrophy (SMA), Friedreich's ataxia, Down's Syndrome, Wemicke-Korsakoff syndrome, and Creutzfeldt-Jakob disease.

18. The method of claim 1, wherein following administration of the stromal cells and the BBB permeabilizing agent, the injured central nervous system tissue has increased expression of synaptophysin, neuronal class III β-tubulin (TUJ1), and doublecortin (DCX1), as compared to an identically injured central nervous system tissue in a mammal that has not been administered stromal cells and a BBB permeabilizing agent.

19. A method of enhancing the cognitive and/or motor functional neurological recovery of a mammal having a central nervous system injury, comprising parenterally administering stromal cells and a blood-brain barrier (BBB) permeabilizing agent to the mammal.

20. The method of claim 19, wherein following administration of the stromal cells and the BBB permeabilizing agent, the cognitive and/or motor functional neurological recovery of the mammal is greater compared to the cognitive and/or motor functional neurological recovery of an identically injured mammal that has not been administered the stromal cells and the BBB permeabilizing agent.

21-32. (canceled)

33. A method of enhancing the engraftment of stromal cells in an injured central nervous system tissue of a mammal having a central nervous system injury, comprising parenterally administering an effective amount of stromal cells and a BBB permeabilizing agent to the mammal.

34. The method of claim 33, wherein the number of stromal cells engrafted in the injured central nervous system tissue, following administration of the stromal cells and BBB permeabilizing agent, is greater compared to the number of stromal cells engrafted in an identically injured central nervous system tissue of a mammal that has not been administered stromal cells and a BBB permeabilizing agent.

35-46. (canceled)

47. A method of treating an injured central nervous system tissue of a mammal having a central nervous system injury, comprising parenterally administering an effective amount of stromal cells and a blood-brain barrier permeabilizing agent.

48. The method of claim 47, wherein the stromal cells have been genetically modified.

49. The method of claim 48, wherein the stromal cells have been genetically modified to increase the expression of a growth factor selected from the group consisting of: nerve growth factor, glial derived neurotrophic factor, ciliary neurotrophic factor, brain derived growth factor, platelet derived growth factor, fibroblast growth factor, and vascular endothelial growth factor.

50-61. (canceled)

62. A composition comprising an effective amount of stromal cells and a BBB permeabilizing agent.

63. The composition of claim 62, wherein the stromal cells have been genetically modified.

64. The composition of claim 63, wherein the stromal cells have been genetically modified to increase the expression of a growth factor selected from the group selected from: nerve growth factor, glial derived neurotrophic factor, ciliary neurotrophic factor, brain derived growth factor, platelet derived growth factor, fibroblast growth factor, and vascular endothelial growth factor.
